(19) United States
(12) Patent Application Publication
(10) Pub. No.: US 2012/0058500 A1
(43) Pub. Date: Mar. 8, 2012

(54) PLATELET AGGREGATION USING A MICROFLUIDICS DEVICE

(75) Inventors: Arnan Deane Mitchell, Victoria (AU); Francisco Javier Tovar Lopez, Victoria (AU); Shaun Phillip Jackson, Victoria (AU); Warwick Scott Nesbitt, Victoria (AU); Josie Carberry, Victoria (AU)

(73) Assignee: Monash University, Clayton (AU)

(21) Appl. No.: 13/255,857

(22) PCT Filed: Mar. 10, 2010
(86) PCT No.: PCT/AU2010/000273

§ 371 Date: Nov. 22, 2011

(30) Foreign Application Priority Data
Mar. 10, 2009 (AU) 2009901033
Oct. 29, 2009 (AU) 2009905303

(51) Int. Cl. C12Q 1/56; C12M 1/34; C12Q 1/34; C12Q 1/02

(52) U.S. Cl. 435/13; 435/29; 435/287.1; 435/288.7; 435/18

(57) ABSTRACT

A microfluidics device to provide real time monitoring of platelet aggregation of a biological sample obtained from a subject. The device comprises a channel configured for passage of the biological sample, the channel comprising a protrusion configured to induce an upstream region of shear acceleration coupled to a downstream region of shear deceleration and defining there-between a region of peak rate of shear, the downstream region of shear deceleration defining a zone of platelet aggregation. The device further comprises a platelet detection means for detecting aggregation of platelets in the zone of aggregation as a result of passage of the biological sample through the channel. Methods to assess real time platelet aggregation of a biological sample obtained from a subject are further described.

### CROSS-REFERENCE TO RELATED APPLICATIONS

The present application claims priority from Australian Provisional Patent Application No 2009901033 filed on 10 Mar. 2009 and Australian Provisional Patent Application No 2009905303 filed on 29 Oct. 2009, the contents of each of which are incorporated herein by reference.

### TECHNICAL FIELD

The present invention relates to a device that facilitates analysis of aggregation of platelets or their progenitors in a biological sample. The device induces localised and controlled disturbances in blood flow which lead to spatially controlled platelet aggregation. The present invention also relates to a method for causing platelets to aggregate in a known location so as to facilitate diagnosis of platelet function and activity. The present invention also relates to a method for controllably modulating the rate and extent of platelet aggregation. The method of the invention is particularly useful for assessing subjects for abnormalities in platelet function. The device is also useful for assaying the function and activity of platelets and their progenitors in response to drug therapy.

### BACKGROUND OF THE INVENTION

Arterial thrombosis remains the single most common cause of morbidity and mortality in industrialised societies. Central to this process is the excessive accumulation of platelets and fibrin at sites of atherosclerotic plaque rupture, leading to vascular occlusion, tissue infarction and organ failure. The heightened thrombogenic potential of advanced atherosclerotic plaques is due to a number of factors; including the high content of tissue factor in the lesion; the presence of potent platelet activating substrates (i.e. collagens); as well as the direct platelet activating effects of high shear stress, caused by narrowing of the vascular lumen by the atherothrombotic process. Rheological disturbances are a cardinal feature of atherothrombosis, with disturbances of blood flow playing an important role in modulating each of the stages of the atherosclerotic process. Atherosclerotic lesions typically develop at arterial branch points or curvature (i.e carotid sinus), where shear rates can be low and flow non-uniform. As lesions progress, luminal stenoses produce a range of flow alterations, such as shear gradients, flow separation, eddy formation and turbulence, each of which can have distinct effects on the atherosclerotic process. The greatest change in blood flow can occur during thrombus development. Flow velocity and shear rates can become extreme with progressive vascular occlusion, establishing a potential dangerous cycle of shear-dependent propagation of the thrombotic process.

Platelet aggregation at sites of vascular injury is of central importance to the arrest of bleeding and for subsequent vascular repair; however an exaggerated platelet aggregation response can lead to the development of arterial thrombi, precipitating diseases such as the acute coronary syndromes and ischemic stroke. There is an increasing appreciation for the importance of hydrodynamic factors in the pathogenesis of vascular disease. However, the precise mechanisms by which rheological changes accelerate the atherothrombotic process remain incompletely understood. Perturbations of blood flow have a significant impact on the adhesion and activation mechanisms of platelets and high shear in particular, can accelerate platelet activation and thrombus growth.

Fluid flow through a tube can be classified as either Newtonian; where the fluid viscosity is independent of fluid shear rates, or non-Newtonian; where the fluid viscosity can change as a function of fluid shear rates. In the case of blood, the cellular components impart a complex viscosity profile that can change dependent on flow rates and vessel geometry and therefore by definition, blood is a non-Newtonian fluid. Under most ex vivo or in vitro conditions blood flow can be considered streamlined or laminar, with adjacent fluid layers travelling parallel to one another. For a Newtonian fluid flowing through a symmetrical vessel the fluid drag at the vessel wall leads to the development of a parabolic flow profile, with maximum flow velocity at the centre of the flow and the minimum velocity at the vessel wall. This hypothetical parallel blood flow arrangement leads to the generation of shear forces between adjacent fluid layers as a result of viscous drag.

The mechanical shear forces imparted by localized blood flow, especially in the case of vascular stenoses at the microscale are complex and diverge significantly from the simple laminar (parallel) flow model. Blood flowing through a stenosed vessel may experience velocity reductions at the entry point to the stenosis, sharp flow accelerations across the stenosis and flow reversal and separations (divergent flow streamlines) at the outlet of the stenosis. These complex rheological conditions may significantly modulate blood platelet function.

Blood platelet aggregation under the influence of blood flow is critically dependent on the adhesive function of both the surface expressed glycoprotein GPIb/V/IX and the integrin family member α_{IIb}β₃ (GP IIb-IIIc). Under conditions of high or elevated shear rates GPIb/V/IX initiates reversible platelet-platelet adhesion contacts while integrin α_{IIb}β₃ stabilizes forming aggregates.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### SUMMARY OF THE INVENTION

According to a first aspect, the present invention provides a microfluidic device to provide real time monitoring of platelet aggregation of a biological sample obtained from a subject, the device comprising:
a channel configured for passage of the biological sample, the channel comprising a protrusion configured to induce an upstream region of shear acceleration coupled to a downstream region of shear deceleration and defining there-between a region of peak rate of shear, the downstream region of shear deceleration defining a zone of platelet aggregation; and
platelet detection means for detecting aggregation of platelets in the zone of aggregation as a result of passage of the biological sample through the channel.

The protrusion may be configured to induce a peak rate of shear within the range 10×10³ s⁻¹ to 150×10³ s⁻¹, when the biological sample is pumped through the device at an appropriate rate which defines and constrains initial shear rates to the physiological range (150 s⁻-˜10,000 s⁻¹). The protrusion may be configured to define and constrain initial shear conditions to within the range 300 s⁻¹-7000 s⁻¹. The protrusion may be configured to define and constrain initial shear conditions to within the range 450 s⁻¹-3,500 s⁻¹. The protrusion may be configured to define and constrain initial shear conditions to about 1,800 s⁻¹. The flow rate may be substantially constant, or may be pulsatile or otherwise varied to change the rate and extent of platelet aggregation.

The protrusion may comprise an upstream face which is at an angle of between 0° to 90° to a dominant direction of flow through the channel to define the region of shear acceleration, and a downstream face which is at an angle of between 0° to 90° to a dominant direction of flow through the channel to define the region of shear deceleration. More preferably the upstream face and downstream face are respectively at an angle of between 30° to 90° to a dominant direction of flow through the channel, and more preferably at an angle of about 85° to a dominant direction of flow. The upstream and downstream faces may be substantially planar, concave or convex.

In one embodiment, the region of peak shear is defined by a gap width with respect to the protrusion and an opposite channel wall, and the gap width is selected from the range 10 μm to 40 μm, for instance, but not limited to 15 μm, 20 μm, 25 μm, 30 μm and 35 μm. A width of the gap, measured parallel to a dominant direction of flow through the channel, is between 0.5 μm and 20 μm.

According to a second aspect, the present invention provides for a microfluidic device for assessing platelet aggregation of a biological sample obtained from a subject, the device comprising:
a channel configured for passage of the biological sample, the channel having a protrusion for perturbing flow of the sample, at least one cross-sectional dimension of the protrusion being less than substantially 100 micrometres, and the protrusion being configured to define a zone of platelet aggregation within the channel; and
platelet detection means for detecting aggregation of platelets at the zone of aggregation as a result of passage of the biological sample through the channel.

In an embodiment of the second aspect, the protrusion may comprise a spherical protrusion located within the channel around which the sample must flow. The spherical protrusion may be centrally located within the channel such that substantially equal amounts of the sample flow on each side of the spherical protrusion.

The protrusion or featuring may comprise a post located within the channel around which the sample must flow. In such embodiments the post may extends from one wall of the channel partially across the channel. In another embodiment, the post is centrally located within the channel such that substantially equal amounts of the sample flow on each side of the post.

In an embodiment of the first or second aspect, a plurality of channels may be provided, each channel having a protrusion of substantially the same dimensions. In such an embodiment the detection means may be operable to detect a sum of all platelet aggregation in all the channels. In one example, the plurality of channels are arranged in parallel. Such embodiments of the invention may be advantageous in improving reliability of detection of platelet aggregation when a single sample is divided and passed through each of the plurality of channels.

In an embodiment of the first or second aspect, a plurality of channels may be provided, each channel having a protrusion of substantially different dimensions. In such an embodiment the detection means may be operable to detect in parallel differential platelet aggregation in the array of channels. In one example, the plurality of channels are arranged in parallel. Such embodiments of the invention may be advantageous in improving screening detection of platelet abnormalities when a single sample is divided and passed through each of the plurality of channels.

In an embodiment of the first or second aspect at least a portion of the channel surface may be provided with a serum protein, an adhesive substrate or a polymer in order to improve platelet aggregation.

In an embodiment of the first or second aspect the channel configuration and flow rate are adapted to maintain Reynolds numbers within the channel less than or equal to about 26, in order to maintain fully stable blood flow without flow separation or vortex formation. In one embodiment a flow rate of 8 microlitres per minute through a microchannel of 20 micrometers diameter yields Reynolds numbers of 0.86, thus ensuring decelerating flow or shear arises without the presence of flow instability or vortex formation.

Any detection apparatus may be used which is capable of detecting and monitoring the platelet aggregation. The detection apparatus may record images of platelet aggregation as a function of time.

In an embodiment of the first or second aspect the present invention further incorporates an optical detection means that may or may not be integrated into the device and may serve as the platelet detection means. The optical detection means may comprise a total internal reflection sensor which is situated adjacent the channel protrusion to monitor real-time platelet aggregation in the zone of platelet aggregation. Optionally, the optical detection means may comprise a light emitter and an aligned light detector, wherein the light emitter is configured to emit light for internal reflection within a material from which the channel is formed, such that the light detector detects changes in internal light reflection brought about by aggregation of platelets in the zone of platelet aggregation. Optionally, the optical detection means may comprise a light emitter and an aligned light detector, and the light emitter is configured to emit light for transmission through the zone of platelet aggregation such that the light detector detects a reduction in transmitted light intensity brought about by aggregation of platelets. Optionally, the optical detection means comprises a light emitter and an aligned light detector, and the light emitter is configured to emit light through a zone of platelet aggregation of each of a plurality of channels as defined by respective protrusions, such that the light detector may detect a reduction in transmitted light intensity brought about by total platelet aggregation in all channels.

The optical detection means and/or means for platelet detection may be configured to observe platelet aggregation in a position away from a sidewall of the channel in order to avoid side wall effects on the platelet behaviour. For example the optical detection means and/or means for platelet detection may be configured to observe platelet aggregation in a position substantially 35 micrometres away from a side wall of the channel.

Optionally, the platelet detection means may comprise a camera. The camera may be a CCD camera. The camera may comprise a radiation direction device, e.g. one or more lens and/or filters and/or mirrors, which directs the radiation from the objects into an image capture element of the camera. The detection apparatus may comprise a microscope. The microscope may detect the object interactions by detecting radiation, e.g. visible light, from the interacting objects. The microscope may operate in a bright field mode and detect radiation comprising visible light. The microscope may operate in a fluorescent mode and detect radiation comprising fluorescent signals. The microscope may be an epi-fluorescent microscope. The microscope may comprise a radiation direction device, e.g. one or more lens and/or filters and/or mirrors, which directs the radiation from the objects into an image capture element of the microscope. The image capture element of the microscope may be a camera.

In an embodiment of the first or second aspect the device may comprise a fabricated block within which are formed, embedded or moulded, one or more fluid-tight channels. The block material may be selected from the group consisting of a polymer, resin, glass, polycarbonate, polyvinyl chloride, or silicon.

In an embodiment, the block material from which the device is fabricated is one of Polydimethylsiloxane (PDMS), borosilicate glass, SF11 glass, SF12 glass, polystyrene and polycarbonate. In a preferred example, the block material is PDMS.

Without wishing to be bound by theory, it is thought that the block material may bind soluble proteins present in the blood sample and that this property of the block material contributes to the effectiveness of the microfluidics device. Accordingly, it is preferable that the block material is of a property that allows soluble blood proteins to bind to the material.

The microchannels of the microfluidics device may be of the same material or a different material to that of the block material. In one embodiment, the cross-sectional diameter of the microchannel is less than 1000 μm. In another embodiment, the cross-sectional diameter is between 100 and 200 μm. In a further embodiment the length of the microchannel is in the range of from about 3 mm to 7 mm, preferably about 5 mm from inlet port to outlet.

The device may comprise an anti-fouling trap situated upstream with respect to the, or each protrusion, to substantially prevent fouling of the respective channels.

The device may further comprise a “solid support” which includes any solid structure having a substantially horizontal surface on which the block may rest. In one embodiment, the solid support may be for example, glass, such as a microscope slide, polymer, polycarbonate, polyvinyl chloride, cellulose or any other optically transparent material.

It will be appreciated that the microfluidics device of the invention can be provided as a disposable or replaceable product or as part of a system.

According to a still further aspect, the present invention provides a system to provide real time monitoring of platelet aggregation of a biological sample obtained from a subject, the system comprising:
a housing;
a microfluidics device according to any one of the embodiments in accordance with the first or the second aspect, the microfluidics device housed within the housing.

The system may comprise a fluid delivery system attached to one or more inlets and/or one or more outlets of the microfluidics device. The fluid delivery system may be attached directly to one or more inlets and/or one or more outlets of the microfluidics device. Optionally the fluid delivery system may be attached indirectly to one or more inlets and/or one or more outlets of the microfluidics device via corresponding inlets and/or outlets of the housing.

The fluid delivery system may be configured to control the flow rate of fluid sample through the, or each, flow channels of the microfluidics device. The fluid delivery system attached to an outlet of the microfluidics device may be a suction pump. The fluid delivery system attached to a sample inlet of the microfluidics device may be a syringe pump, gravity feed, peristaltic pump or any form of pressure driven pump. The suction and pressure pumps can be a powered pump or a manually operated pump (such as a syringe).

The system may further comprise a heater which supplies heat to the microfluidics device. The heater may be provided in or attached to a platform on which the microfluidics device is placed. The heater may comprise resistive electrical coils, a printed pattern of resistive ink, or the like. The heater may be a resistive heater comprising a serpentine wire coated with a thermally conductive adhesive. The heater may be capable of regulating the temperature of sample fluid in the microfluidics device within the range 37° C. to 60° C., preferably around 37° C.

The system may comprise software integrated within the system to allow control of the various parts of system, for example temperature control of a platform on which the microfluidics device is located, pump control of injection of fluid into the device, calculations of flow rate within the device, control of the camera configuration such as capture parameters, and image processing. Each of these control areas may be modularised and may be used independent of, or in conjunction with, a main control processor.

The system may comprise a positioning apparatus to position the microfluidics device relative the detection apparatus.

The optical detection apparatus may further incorporate means for recording the aggregation of platelets. When images are recorded by the detection apparatus, a number of images at different time points may be recorded in order to determine the extent of platelet aggregation observed in real time in the microfluidics device.

Visualisation of objects may be enhanced, and platelet aggregation more readily determined, by labeling the objects in the biological sample with a colour or fluorescence marker. Thus, the method may include the step of mixing a colour or fluorescence marker with the biological sample. This step may be carried out prior to, during, or after the step of providing the biological sample to the channel. For example, the biological sample may be mixed with the colour or fluorescence marker:—outside of the device prior to the biological sample being introduced into the sample inlet; between the sample inlet and the flow cavity (for example in a mixing well provided in the passage between the inlet and the flow cavity).

Examples of suitable fluorescent markers that can be used according to the invention include for example, long chain carbocyanines such as DiI, DiO and analogs. Specific examples include the lipophilic carbocyanines DiIC₁₈, DiIC6, DiOC₁₈, DiOC₆, which are manufactured by Invitrogen as well as membrane probes manufactured by Sigma.

Other membrane probes that are suitable for use in the present invention will be familiar to persons skilled in the art.

When visualising objects that include a fluorescent marker, the method may include the step of shining radiation from an excitation radiation source onto the labelled platelets to excite the fluorescence marker. The radiation may be shone onto the platelets through appropriate excitation filters. The excitation radiation source may comprise part of the overall system of the invention. The excitation radiation source may for example be a blue-light emitting source, such as a diode or other suitable source. The detection apparatus may comprise emission filters, positioned such that the source directs radiation to pass there through before arriving at the device.

According to a third aspect the present invention provides a method to assess real time platelet aggregation of a biological sample obtained from a subject, the method comprising:
passing the biological sample through a featured channel at a rate which causes the channel featuring to perturb flow of the sample so as to induce an upstream region of shear acceleration coupled to a downstream region of shear deceleration and defining there-between a region of peak rate of shear, the downstream region of shear deceleration defining a zone of platelet aggregation; and
detecting aggregation of platelets in the zone of aggregation as a result of passage of the biological sample through the channel.

The featured channel is to be understood to comprise a protrusion as described above in relation to the first aspect, the second aspect, or any one of its embodiments.

The present invention also provides a method for assessing platelet aggregation of a biological sample obtained from a subject, the method comprising:
passing the biological sample through a channel having a protrusion for perturbing flow of the sample, at least one cross-sectional dimension of the protrusion being less than substantially 100 micrometres, the protrusion being configured to define a zone of platelet aggregation within the channel; and
detecting aggregation of platelets at the zone of aggregation as a result of passage of the biological sample through the channel.

The present invention also provides a device for assessing platelet aggregation of a biological sample obtained from a subject, the device comprising:
a channel configured for passage of the biological sample, the channel being featured in a manner to perturb flow of the sample so as to induce a high shear zone in the sample when passed through the channel at an appropriate flow rate and to induce a zone of platelet aggregation in a region of negative shear gradient downstream of the high shear zone; and
platelet detection means for detecting aggregation of platelets at the zone of aggregation as a result of passage of the biological sample through the channel.

The present invention also provides a method for assessing platelet aggregation of a biological sample obtained from a subject, the method comprising:
passing the biological sample through a featured channel at a rate which causes the channel featuring to perturb flow of the sample so as to induce a high shear zone in the sample and to induce a zone of platelet aggregation in a region of negative shear gradient downstream of the high shear zone; and
detecting aggregation of platelets at the zone of aggregation as a result of passage of the biological sample through the channel.

In some embodiments of the invention, prior to sample perfusion, degassed Tyrodes buffer (4.3 mM K₂HPO₄, 4.3 mM NaHPO₄, 24.3 mM NaH₂PO₄, 113 mM NaCl, 5.5 mM D-glucose, pH 7.2) is used to prime the channels to remove any bubbles. Typically, the Tyrodes buffer is heated to 45° C.

The protrusion or featuring may comprise a barrier partially obstructing the channel. In such embodiments, a gap between the barrier and an opposite channel wall is preferably substantially between 0.5 and 40 micrometres. A width of the gap, measured parallel to a dominant direction of flow through the channel, is preferably between 0.5 and 20 micrometres and more preferably about 15 micrometres, and is preferably configured to yield shear conditions of around 20,000 s⁻¹ under suitable flow rates. However it is to be appreciated that the peak shear rates may be in the range of substantially 10,000 s⁻¹ to 150,000 s⁻¹ or more. An input channel is preferably configured to produce shear conditions of around 1,800 s⁻¹ upstream of the gap. The barrier preferably comprises an upstream face which is at an angle of between 30 degrees and 90 degrees to a dominant direction of flow through the channel. The barrier preferably further comprises a downstream face which is at an angle of between 30 degrees and 90 degrees to a dominant direction of flow through the channel. The upstream and downstream faces may be substantially planar, concave or convex. In one embodiment, the device further comprises an inlet or aperture for accepting the biological sample and an outlet. The inlet and outlet are situated at either end of each microcapillary or microchannel in connection therewith.

Typical flow rates contemplated herein cover the range required to develop those proposed to exist in the vasculature in vivo. Typically, the flow rate of the biological sample through the microcapillary or microchamber is in the range of 500-0.5 microlitres per minute, and for example may be in the range of 2 to 42 μl/min.

The present invention also provides a diagnostic method for the detection or assessment of a subject who has, or is at risk of developing, a condition or disorder involving abnormal function or activity of platelets or their progenitors; said method incorporating the microfluidics device of the present invention.

The invention also provides a method for diagnosing in a subject, the presence of, or risk of developing, a condition or disorder involving abnormal function or activity of platelets or their progenitors, comprising:
i) obtaining a biological sample from the subject;
ii) passing the biological sample through the device according to the invention under defined flow conditions and for a time sufficient to enable cells from the biological sample to aggregate;
iii) detecting any aggregation of said cells; and
   comparing the time to and size of the aggregation of cells of the biological sample with a predetermined standard, wherein any variation is indicative of the presence of or risk of developing a condition or disorder involving abnormal function or activity of platelets or their progenitors.

In one embodiment of the invention, the method may be used to diagnose thrombus development and dissolution, cardiovascular disease, changes to haemostatic mechanisms due to disease and drugs, platelet dysfunction and receptor abnormality, sensitivity to drug therapy, bleeding disorders such as, Von Willebrand disease or vitamin K deficiency, stenosis, diabetes mellitus, clotting disorders, stroke risk, or platelet function disorders such as Glaanzman's Thrombasthenia, Bernard-Soulier syndrome, and Storage Pool Disease.

By “abnormal function or activity of platelets” it is meant any activity or defect associated with platelet adhesion, platelet aggregation, platelet translocation, platelet velocity, platelet morphology, and thrombus stability. The term is also intended to include any defect in platelet degranulation and release of cytoplasmic granules. The term is also intended to include abnormalities in plasma factors affecting platelet function.

Various platelet defects will be known to persons skilled in the art of the present invention.

The present invention also provides a method for determining or assessing the modulating effect of a reagent(s) on the aggregation of platelets or their progenitors in a biological sample, the method comprising:
i) passing said biological sample in the presence of said reagent(s) through the microfluidics device of the invention, under defined flow conditions and for a time sufficient to determine whether platelet aggregation has occurred within said device; and
ii) comparing the result obtained in step (i) with the result when step (i) is performed in the absence of said reagent(s).

It will be appreciated that the device and methods of the invention can be used to assess the effectiveness of anti-platelet agents in subjects treated with anti-platelet drugs. Such subjects include those treated by interventional cardiology catheterization. This includes angiograms, angioplasty, and stent placement. In addition, the device can be used to monitor the effectiveness of anti-platelet agents in patients who receive an artificial heart valve.

The device and methods of the invention can be used to assess the effectiveness of asprin or other anti-platelet agents in subjects taking the agents to prevent a cardiovascular event, such as a coronary thrombosis (heart attack), pulmonary embolism, stroke, or deep vein thrombosis due to excessive platelet activity.

The device and methods of the invention can also be used to diagnose subjects for their risk of excessive bleeding. This testing can be needed, for instance, prior to a surgical or dental procedure. For example, the methods can be used on patients prior to having a tooth pulled or wisdom tooth removed to determine their risk of excessive bleeding.

The present invention also provides for the use of the microfluidics device of the invention in a method for diagnosing a subject who has, or is at risk of developing, a condition or disorder involving abnormal function or activity of platelets or their progenitors.

According to one embodiment, the reagent(s) may be added to the biological sample prior to perfusion through the device. Alternatively, the reagent(s) may be administered to the subject prior to the biological sample being taken from the subject.

In another example, the reagent(s) may be applied to the walls of the microchannel and thus added to the biological sample as it passes through the microchannel of the device.

For example, in the case of a blood sample taken from a patient on the antiplatelet drug clopogrel, the biological sample could be pre-treated with the reagent P2Y1 (ADP) receptor antagonist MRS2179 in order to sensitise the system to the effects of clopidogrel.

The choice of appropriate dose of reagent to pre-treat the sample prior to perfusion through the microfluidics device will be known to persons skilled in the art. The inhibitor concentration to be used in the pre-treatment may be determined by standardised platelet aggregometry in response to exogenous ADP addition to the platelet sample, fluorescence activated cell sorting based on the activation of the platelet integrin α_{IIB}β₃ by exogenous ADP addition to the platelet sample, or via dose response measurements in various iterations of the microfluidics device itself

The invention also provides a method of monitoring the treatment of a subject undergoing therapy with a reagent, the method comprising:
(i) passing a first biological sample from the subject through the device of the invention, under defined flow conditions and for a time sufficient to determine whether platelet aggregation has occurred within said device, said first biological sample being obtained prior to administration of the reagent to the subject, and
(ii) passing a second biological sample from the same subject through the device of the invention, under defined flow conditions and for a time sufficient to determine whether platelet aggregation has occurred within said device, said second biological sample being obtained after administration of the reagent to the subject; and
(iii) comparing the result obtained in step (i) with the result obtained in step (ii).

In another embodiment according to the invention, the first and second samples are both obtained after administration of the reagent to the subject so that the effect of the reagent can be monitored over time. For example, the second biological sample may be taken at a defined period of time after the first sample, for example, after 1 day, after 5 days, after 1 week, after 1 month, after 4 months in order to progressively monitor the patient's therapy.

Accordingly, the invention also provides a method of monitoring the treatment of a subject undergoing therapy with a reagent, the method comprising:
(i) passing a first biological sample from the subject through the device of the invention, under defined flow conditions and for a time sufficient to determine whether platelet aggregation has occurred within said device, said first biological sample being obtained after a first dose of the reagent to the subject, and
(ii) passing a second biological sample from the same subject through the device of the invention, under defined flow conditions and for a time sufficient to determine whether platelet aggregation has occurred within said device, said second biological sample being obtained after a second dose of the reagent to the subject; and
(iii) comparing the result obtained in step (i) with the result obtained in step (ii).

By comparing the platelet aggregation behaviour over time after treatment with the reagent, it will be possible for the clinician to modify the dose of the reagent that is being administered to the subject as well as make an informed decision as to whether to discontinue therapy with the reagent or otherwise change the reagent being administered.

The invention also provides for the use of the device according to the invention for monitoring anti-platelet therapy in a subject. In one example, the device may be used to identify subjects displaying asprin and clopidogrel “resistance” or other manifestations of treatment failure.

The term “for a time sufficient to determine whether platelet aggregation has occurred” will be a period of time that the biological sample flows through the device and such period will be familiar to persons skilled in the art of the present invention. In one example, the period of time is at least about 10 mins. In another example it is at least about 20 mins.

The invention also provides for the use of the microfluidics device according to the invention to monitor platelet function and/or viability in a biological sample.

For example, the device may be used to screen and act as a form of quality control for platelet isolates and preparations used for clinical treatment (e.g. infusion) of patients suffering from platelet related bleeding disorders. The device may also be used to assess the viability and effectiveness of platelet transfusion products prior to administration into a patient. The device may also be used to assess the viability and effectiveness of platelets following prolonged storage.

The invention also provides for the use of the microfluidics device according to the invention as a screen for bleeding disorders.

In one example, a biological sample obtained from a subject may be pre-treated with one or more platelet inhibitors and passed through a number of defined geometries of the microfluidics device where the extent of platelet aggregation observed in the device can be correlated with a bleeding disorder.

The device can be used to determine the causes of bleeding in both congenital (e.g. von Willebrand's disease) and acquired bleeding defects (e.g. drugs, acquired thrombocytopathies). Congential bleeding disorders may include the following:
von Willebrand's disease, Glanzmann Thrombasthenia, Bernard-Soulier Syndrome, Scott Syndrome;
α-granule Defects such as Gray Platelet Syndrome, Quebec Platelet Syndrome, α-SPD (storage pool defects), α,δ-SPD;
Dense Granule Defects such as Hermansky-Pudlak Syndrome, Chediak-Higashi Syndrome, Griscelli Syndrome, δ-SPD;
Cytoskeletal Defects such as Wiskott-Aldrich Syndrome and MYH9 and associated giant platelet disorders.

The device may also be used to assess patient-to-patient differences in drug response, and can be used to identify patients who are at high risk for bleeding.

The invention also provides for the use of microfluidics device according to the invention for the analysis of bleeding disorders in paediatric subjects. For example, the device may be used for screening the neonatal and paediatric population of patients where only small samples of blood are available. In another example, the device may be used to detect infants and/or neonates at risk of intracranial haemorrhage. The device may be used to establish if bleeding is principally related to platelet dysfunction.

In other embodiments of the invention, the invention could incorporate an array of varying geometries in parallel ranging between 6-300 geometric variations as a first pass assay device. The results from this broad spectrum array could then be used to define a specific set of geometries most appropriate to the platelet sample in question. This could be viewed as a calibration step that focuses the assay on a subset of geometries. The array versions of the device find utility in high throughput screening protocols.

Accordingly, the invention also provides for the use of the microfluidics device of the invention as an experimental high throughput screening tool for drug development of anti-platelet therapies. In one embodiment, a plurality of platelet samples are treated with a range of small molecule or peptide inhibitors and analysed by passing the sample through the microfluidics device. In this way, novel anti-platelet drugs may be identified from large chemical libraries quickly and efficiently. Molecules or peptides with anti-platelet activity would be analysed and compared with untreated control samples perfused through a defined series of microchannel geometries.

The invention also provides a method for high throughput screening of a plurality of candidate anti-platelet compounds, the method comprising:
(i) contacting at least one biological sample obtained from a subject with at least a first member of the plurality of candidate anti-platelet compounds;
(ii) passing the sample through the microfluidics device of the invention, under defined flow conditions and for a time sufficient to determine whether platelet aggregation has occurred within said device;
(iii) detecting an effect of the first member of the plurality of candidate anti-platelet compounds on the platelet aggregation of the at least one biological sample; and
(iv) comparing the effect observed in (iii) with a control sample that has not come into contact with the candidate compound.

It will be appreciated by persons skilled in the art of the present invention, that the candidate anti-platelet compound may comprise a detectable labelling group to facilitate the detection and observation of platelet aggregation in the device.

It will also be appreciated that the above high throughput screening method may be advantageous as a screening tool for screening a plurality of platelet samples derived from transgenic animals such as transgenic mice for shear dependent platelet defects. The high throughput array version of the device would allow for large numbers of samples from mice that have undergone recombinant or chemical mutation to be screened rapidly for platelet defects. The method may also be used to screen samples for a large number of transgenic mice.

The invention also provides for a novel anti-platelet reagent, said reagent obtained by high throughput screening incorporating the microfluidics device according to the invention.

The invention also provides a kit for use in monitoring platelet function, comprising packaging material comprising:
(i) a microfluidics device according to the invention; and
(ii) instructions for indicating that the device is to be used in a system for monitoring platelet function.

The present embodiments have been developed in recognition that local shear micro-gradients promote platelet aggregation at a zone where shear deceleration occurs immediately following a zone of high shear acceleration. Thus, a zone of shear acceleration followed by a tightly coupled zone of decelerating shear (shear gradient) is a condition conducive to the development of stabilised platelet aggregates.

### BRIEF DESCRIPTION OF THE DRAWING

An example of the invention will now be described with reference to the accompanying drawings, in which:

FIG. 1 is a schematic generally illustrating flow of a sample past a spherical protrusion which defines a zone of platelet aggregation;

FIG. 2a is a micrograph sequence illustrating platelet aggregation at and downstream of a vascular injury, FIGS. 2b and 2c illustrate an extent of platelet aggregation in three zones about the vascular injury, and FIG. 2d illustrates the extent of platelet aggregation as a function of shear rate;

FIG. 3a is sequence of differential contrast images, and FIG. 3b comprises scanning electron microscope images, each illustrating platelet tethering;

FIG. 4a is a perspective view generally illustrating a channel having a barrier in accordance with one embodiment of the invention, FIG. 4b is a top view illustrating variable channel parameters which may be selected in some embodiments of the invention, and FIG. 4c is a micrograph of a fabricated device in accordance with a second embodiment of the present invention;

FIG. 5a is a cross sectional end view of a block within which a barrier step geometry micro-channel has been fabricated in accordance with an embodiment of the present invention, FIG. 5b is an enlarged partial end view of the channel portion of the block of FIG. 5a, FIG. 5c is a top view of the block of FIGS. 5a and 5b, and

FIG. 5d is an enlarged partial top view of the block of FIGS. 5a-5c;

FIG. 6a illustrates an embodiment of the invention in which the protrusion comprises a sphere in the channel, while FIGS. 6b to 6d illustrate variations on such sphere geometries; FIG. 7a is a cross sectional end view of a block within which a sphere geometry micro-channel has been fabricated in accordance with another embodiment of the present invention, FIG. 7b is an enlarged partial end view of the channel portion of the block of FIG. 7a, FIG. 7c is a top view of the block of FIGS. 7a and 7b, FIG. 7d is an enlarged partial side view of the block of FIGS. 7a-7c, and FIG. 7e is an enlarged partial top view of the block of FIGS. 7a to 7d;

FIG. 8 illustrates an embodiment of the invention in which the protrusion comprises a post in the channel;

FIG. 9a is a cross sectional end view of a block within which a post geometry micro-channel has been fabricated in accordance with a further embodiment of the present invention, FIG. 9b is an enlarged partial end view of the channel portion of the block of FIG. 9a, FIG. 9c is a top view of the block of FIGS. 9a and 9b, FIG. 9d is an enlarged partial side view of the block of FIGS. 9a-9c, and FIG. 9e is an enlarged partial top view of the block of FIGS. 9a to 9d;

FIG. 10a is a perspective view of a polydimethylsiloxane (PDMS) block into which a micro-channel device in accordance with one embodiment of the present invention has been fabricated, FIG. 10b illustrates several differential interference contrast images showing several physical embodiments of the device design in a parallel array configuration; and FIG. 10c is a top view of a PDMS block into which a micro-channel device in accordance with a further, preferred, embodiment of the present invention has been fabricated;

FIGS. 11a to 11e illustrate results obtained in a first example of the invention;

FIGS. 12a and 12b illustrate results obtained in a second example of the invention;

FIGS. 13(i) and 13 (ii), each of which show images (a) to (f) illustrate colour and black and white images respectively of results obtained in a third example of the invention;

FIG. 14a illustrates platelet aggregation in three channel microgeometries in which the expansion angle b differs and takes the values of 90 degrees, 60 degrees and 30 degrees, respectively, for uninhibited whole blood. A=c90 e90 g20 w15 100-700 μm geometry, B=c90 e60 g20 w15 100-700 μm geometry, and C=c90 e30 g20 w15 100-700 μm geometry.

FIG. 14b illustrates platelet aggregation in the same three geometries for whole blood treated with inhibitors. A=c90 e90 g20 w15 100-700 μm geometry, B=c90 e60 g20 w15 100-700 μm geometry, and C=c90 e30 g20 w15 100-700 μm geometry.

FIGS. 15a-15d illustrate strain rate and acceleration analysis for a selection of step geometries;

FIGS. 16a-d show structural and CFD simulations of a representative mouse mesenteric arteriole undergoing side wall compression and FIGS. 16e-f show black and white illustrations corresponding to 16a-b;

FIG. 17 describes three selected symmetric micro-channel design cases;

FIGS. 18(i) and 18(ii), each of which show images (a) to (d) illustrate colour and black and white images respectively of computed strain rate distributions in the mesenteric arteriole and the c60g20e60 vascular mimetic;

FIGS. 19(i) and 19(ii), each of which show images (a) to (d) illustrate colour and black and white images respectively of hydrodynamic performance of the device;

FIGS. 20a and 20b show colour and black and white images respectively of real-time epi-fluorescence images showing aggregation;

FIGS. 21a-b show a series of test-case experiments in which both the contraction and expansion angles of the microchannel geometry were symmetrically modified, FIGS. 21c-d show show black and white illustrations corresponding to 21a-b;

FIG. 22 shows a comparison of the anti-platelet inhibitor effects in a microfluidics device containing a c85 g30 e85 100-100 μm geometry.

FIG. 23 shows a comparison of a normal health donor sample versus a von Willebrand disease patient sample in a microfluidics device containing a c85 g30 e85 100-100 μm geometry.

FIG. 24 shows a comparison of decreasing contraction angle on the platelet aggregation response in a microfluidics device containing a cX g20 e85 100-100 μm geometry, where cX=contraction angle.

FIG. 25 shows a comparison of decreasing expansion angle on the platelet aggregation response in a microfluidics device containing a c85 g20 eX 100-100 μm geometry, where eX=expansion angle.

FIG. 26 shows an analysis of the gap width on the platelet aggregation response in a microfluidics device containing a c75 gX e75 100-100 μm geometry, where gX=variable gap width.

FIG. 27 shows an analysis of the gap length on the platelet aggregation response in a microfluidics device containing a c75 g20 e75 100-100 μm geometry.

### DETAILED DESCRIPTION OF THE INVENTION

The present inventors have identified a key role for sudden alterations in blood rheology in initiating platelet aggregation and thrombus growth at sites of vascular injury. In particular, the present inventors have demonstrated a critical role for micro-scale shear gradients in inducing discoid platelet aggregation, with stabilization of aggregates dependent on the development of a unique membrane adhesion structure, termed membrane tether restructuring. Thus in response to localised shear micro-gradients, developing thrombi are principally composed of discoid platelets, with the generation of soluble platelet agonists, such as thrombin, ADP and TXA2, playing a secondary role in stabilising formed aggregates. These new findings challenge the long-held view that soluble agonist generation is the principal driver of platelet aggregation and thrombus growth.

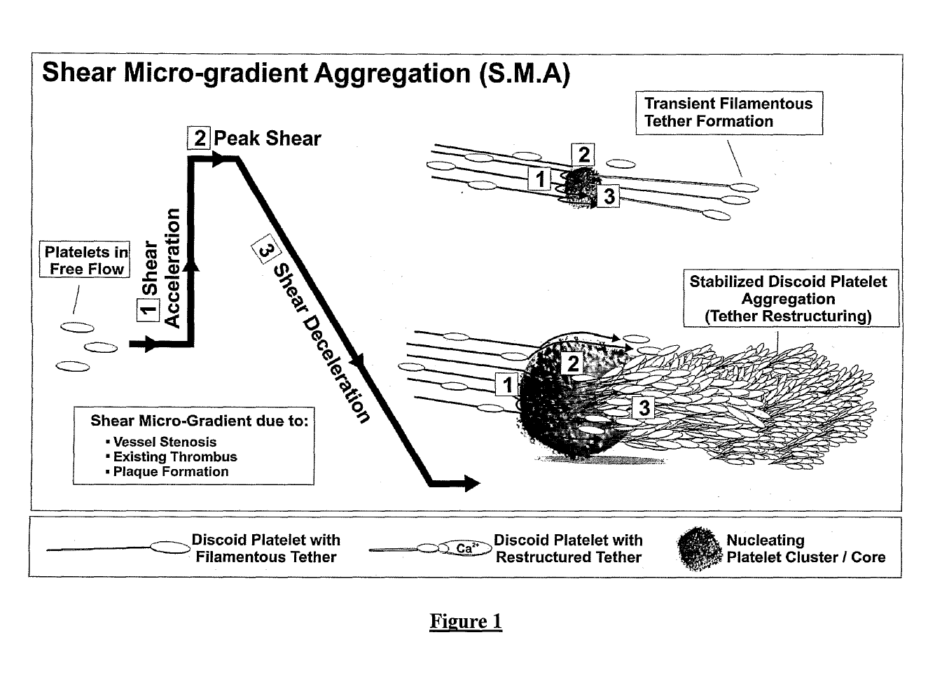

FIG. 1 is a schematic generally illustrating flow of a sample past a spherical protrusion which defines a zone of platelet aggregation. This shows a working model of shear gradient -dependent platelet aggregation (S.G.A) that underpins the micro-shear gradient technology described further in the following. Localized perturbation of blood flow due to changes in vessel wall geometry or as a result of partial luminal obstruction i.e. by a developing thrombus, establishes a local shear gradient typified by a narrow zone of shear acceleration followed by a tightly coupled zone of shear deceleration. Discoid platelets following path-lines that intersect the zone of shear acceleration form filamentous membrane tethering interactions within the peak shear region (Zone 2). Subsequent translocation of these platelets into zones of decelerating shear (Zone 3) leads to an active (Ca²⁺-dependent) restructuring of membrane tethers, characterized by overall tether thickening and adhesion strengthening. Ongoing discoid platelet recruitment and tether restructuring promotes stabilized discoid aggregates and thrombus propagation downstream from the site of vascular injury.

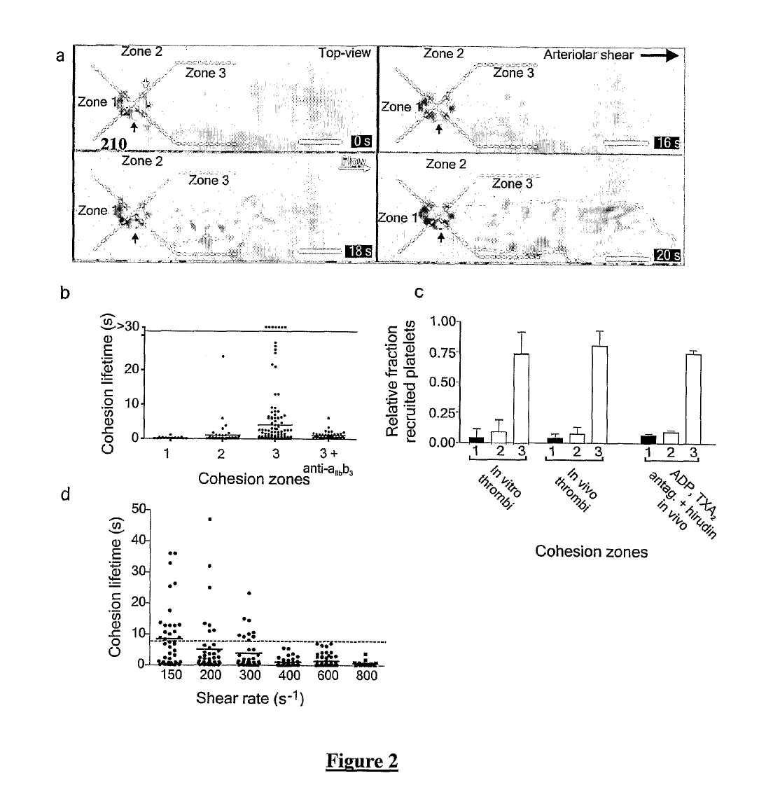

FIG. 2a is a representative micro-imaging sequence showing discoid platelet aggregation occurring at a site of chemical damage to the wall of a mesenteric arteriole in a mouse. Note the growing platelet aggregate has been nominally segmented into an upstream quadrant (zone 1), lateral quadrants (zones 2) and a downstream quadrant (zone 3). Black arrows indicate the lesion caused by chemical treatment. White arrows indicate the point at which initial platelet recruitment was observed. Broken white line demarcates the outer margin of the discoid platelet aggregate. Scale bar 5 μm. FIG. 2b provides a graph showing discoid platelet cohesion lifetimes in differing shear zones (zones 1, 2 and 3) at the surface of a platelet thrombus in vitro (n=24 experiments). Note that cohesion lifetime is significantly greater in the low shear zone (zone 3).

FIG. 2c is a graph showing the relative fraction of discoid platelets tethering within the differing shear zones (zones 1, 2 and 3) of developing murine thrombi in vitro and in vivo; In vitro thrombi-cohesion frequency at the surface of in vitro thrombi (n=24 experiments); In vivo thrombi—cohesion frequency at the surface of in vivo thrombi in C57B1/6 wild-type mice.; ADP, TXA₂ antagonists.+hirudin in vivo—cohesion frequency at the surface of in vivo thrombi in P2Y₁^{-/-} mice administered with 200 mg/kg aspirin, 50 mg/kg clopidogrel orally+intravenous hirudin (50 mg/kg) (n=14). Note that the principle region of platelet recruitment occurs within the deceleration zone (zone 3).

FIG. 2d is a graph showing discoid platelet cohesion lifetimes at the surface of preformed platelet monolayers in vitro as a function of applied bulk shear rate (γ) [n=3]. This data set demonstrates that platelet recruitment through tether formation is most efficient at or below a shear rate of 300 s−1 approaching that found within zone 3 of in vitro and in vivo thrombi.

Overall this data set demonstrates that resting discoid platelet adhesion and cohesive interactions are sensitised to regions of shear deceleration that occur at the downstream face of in vitro and in vivo thrombi. This is the fundamental observation that forms the biological basis of the device design and as published in Nesbitt W. S. et. al., “A shear gradient-dependent platelet aggregation mechanism drives thrombus formation”. Nat Med. 2009 Jun; 15 (6):665-73.

FIG. 3a is a sequence of differential contrast images, and FIG. 3b comprises scanning electron microscope images, each illustrating the dynamic structural rearrangement of blood platelets as a function of micro-shear gradient application. FIG. 2a illustrates differential interference contrast (DIC) imaging showing dynamic platelet tether behaviour at the downstream face of a thrombus, preformed on an immobilized Type 1 fibrillar collagen (applied bulk shear rate=1800.s⁻¹) [Scale bar=2 μm]. The white marquee highlights the progression of a discoid platelet tether: Initial platelet interaction results in the formation of a short tether (144 sec) that rapidly thickens (161-188 sec) to produce a bulbous membrane structure proximal to the discoid body (white arrow: 191 sec). FIG. 2b illustrates scanning electron microscope (SEM) imaging of discoid platelets exhibiting filamentous and restructured membrane tethers during adhesion to the surface of spread platelet monolayers (flow at 300.s⁻¹) [Scale bar=1].

The research leading to the identification of this novel shear gradient dependent platelet aggregation mechanism has resulted in the development of a microfluidies-based flow device that utilises temporal shear gradients to induce platelet aggregation and thrombus development.

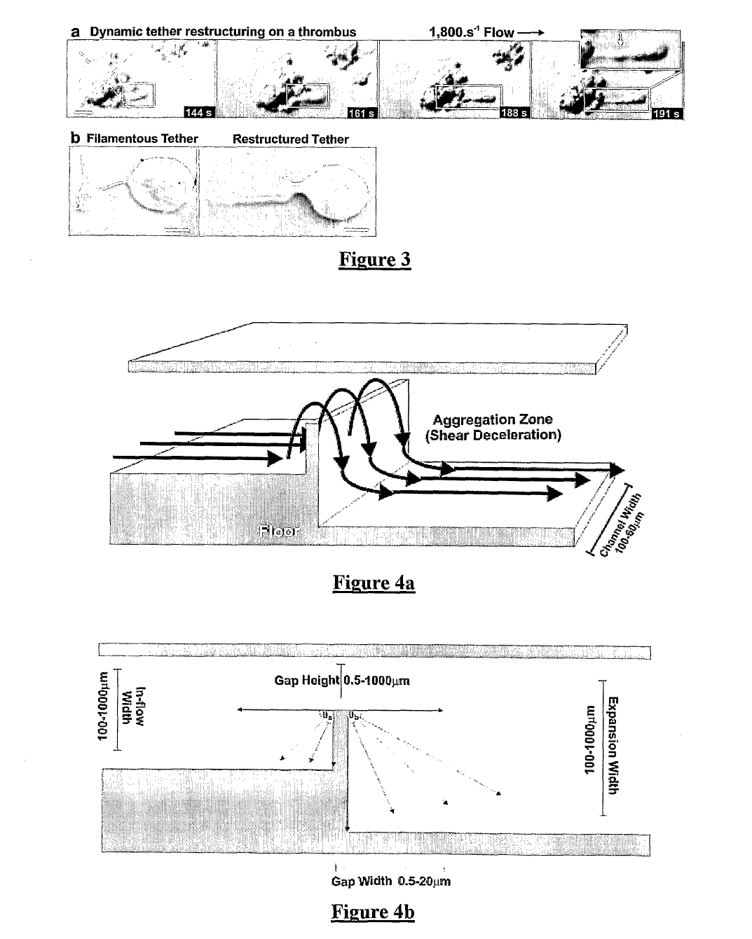

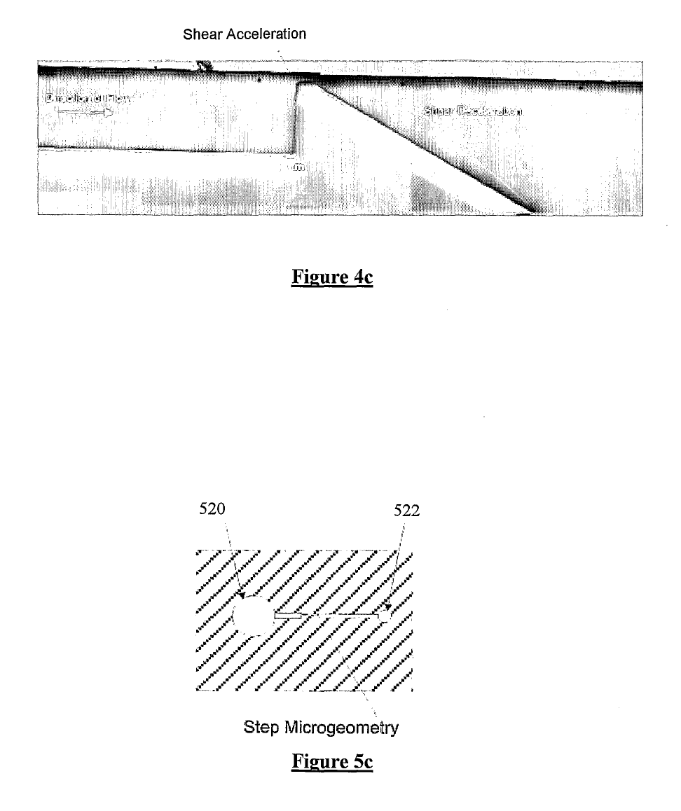

FIGS. 4a to 4c respectively illustrate a schematic of a micro-shear gradient device having a step geometry. FIG. 4a is a schematic of the micro-shear gradient device depicting the overall principle of the step geometry configuration. A blood sample is perfused from left to right through the micro-shear gradient chamber. Interaction of the sample with the microscale step geometry leads to initial shear acceleration over the barrier, followed by a tightly coupled deceleration phase immediately downstream of the barrier and step, that drives the aggregation of discoid blood platelets within the aggregation zone. FIG. 4b illustrates that, in this embodiment, the step geometries are defined by 6 principal parameters: i. The in-flow channel width (100-1000 μm) which defines and constrains initial blood shear rates to the physiological range (150-10,000.s−1); ii. The in-flow angle, or contracting angle (θ_{c}) ranging from 0° through 90° (but more preferably 30° through 90°) that defines the rate of blood flow acceleration; iii. the step gap height ranging from 10 μm to 40 μm which defines the peak shear following the acceleration phase; iv. the expansion angle (θₑ) ranging from 0° through 90° (but more preferably 30° through 90°) that defines the critical rate of blood flow deceleration into the expansion geometry, defining the zone of platelet aggregation; v. the expansion channel width ranging from 100-1000 μm that defines the magnitude of the deceleration phase and vi. the gap width ranging from 0.5-20 μm which defines the width of the protrusion. FIG. 4c is a micrograph (40× magnification) showing a fabricated micro-shear gradient device consisting of an in-flow width of 100 μm, θc=90°, gap height of 10 μm, θe=30°, and an expansion width of 700 μm (only partially visible in Figure).

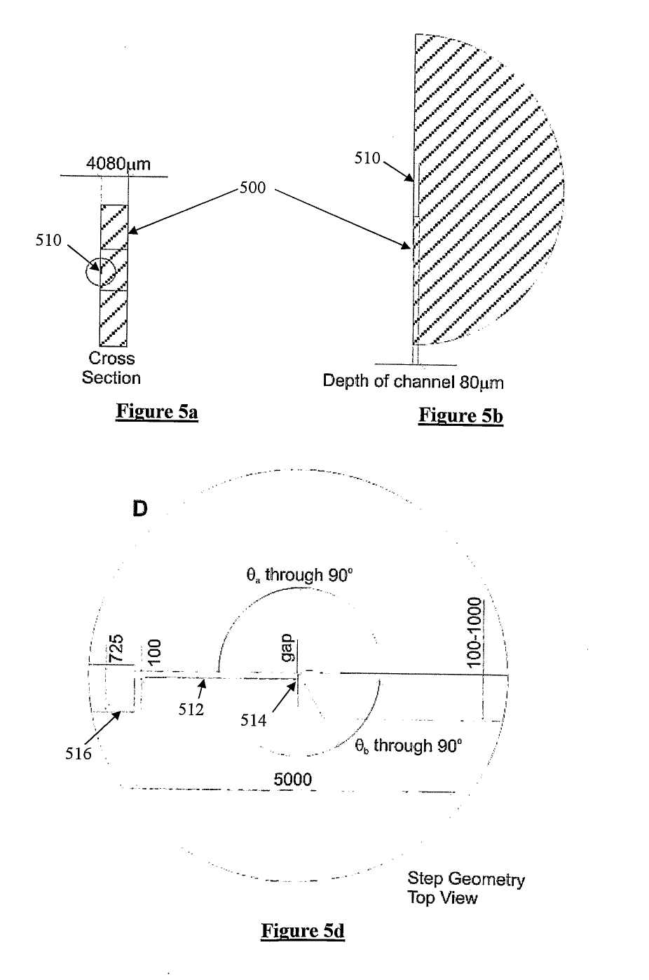

FIGS. 5a to 5d respectively illustrate a schematic of a micro-shear gradient device having a step geometry of the type portrayed in FIG. 4. FIGS. 5a. & 5b give cross sectional views of the microchannel polydimethylsiloxane (PDMS) block 500, showing the position and dimensions of the rectangular microchannel 510. FIG. 5c is a top view of the microchannel device 500 with step geometry, showing the inlet port 520 of diameter 16 mm, and outlet port 522 of diameter 2 mm. FIG. 5d is a detailed top view schematic of the step geometry of block 500, showing the position of the step geometry relative to the microchannel 512. As shown in FIG. 5d, the feed channel 516 from inlet port 520 is of width 725 micrometres, microchannel 512 is of width 100 micrometres, the barrier of step 514 leaves a gap of width between 10 μm to 40 μm at a downstream end of the microchannel 512, and the outflow channel 518 is of a width in the range of 100-1,000 micrometres. An upstream face of the barrier of the step 514 presents an angle θ_{c} to the flow direction selected between 0 and 90 degrees (but more preferably 30° through 90°), and the downstream face presents an angle θₑ selected between 0 and 90 degrees (but more preferably 30° through 90°).

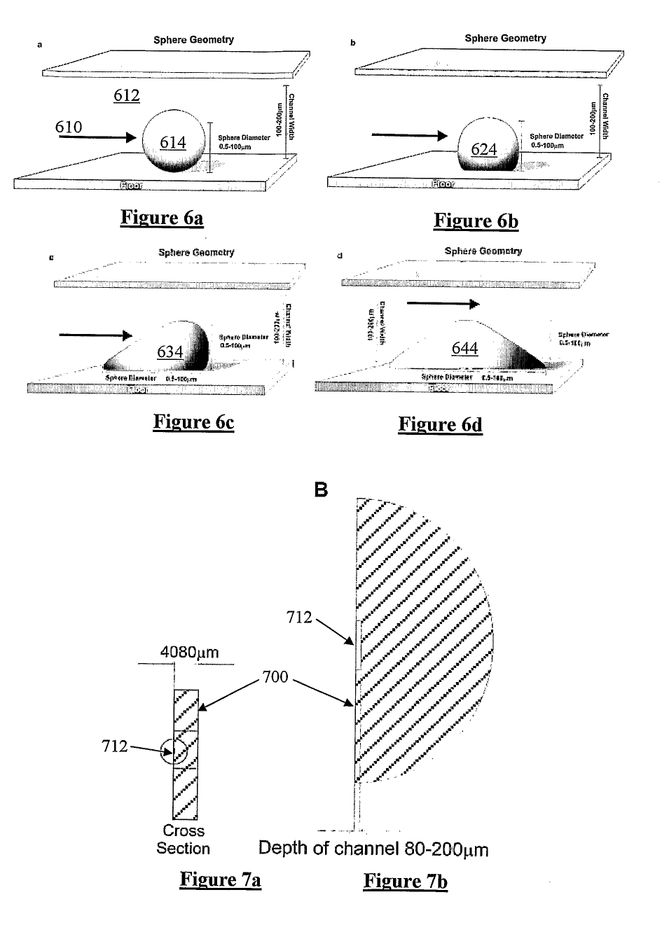

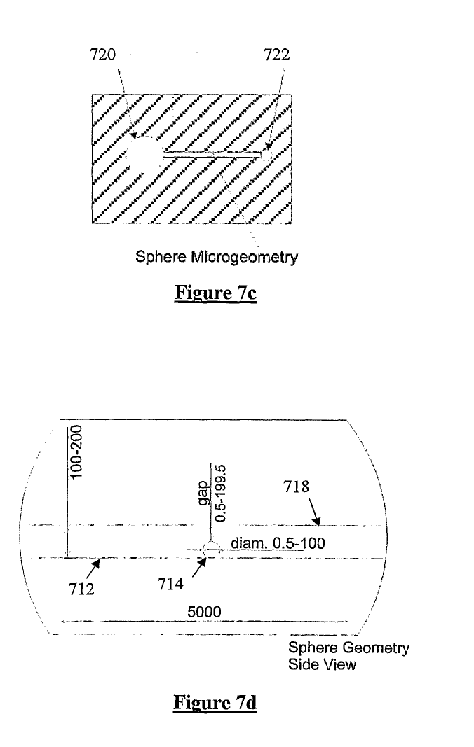

FIGS. 6a to 6d illustrate embodiments of the micro-shear gradient device in which a sphere geometry is used. FIG. 6a is a schematic of a micro-shear gradient device, depicting the overall principle of the sphere geometry configuration. Arrow 610 indicates that a blood sample is perfused from left to right through the micro-shear gradient chamber 612. Interaction of the sample with the microscale sphere geometry 614 leads to lateral and axial shear acceleration immediately upstream of the sphere 614 followed by a tightly coupled deceleration phase immediately downstream of the sphere 614, the latter driving the aggregation of discoid blood platelets at the downstream face of the sphere geometry 614. The sphere geometries are defined by 2 principal parameters: i. The channel width (100-200 μm) which defines and constrains initial blood shear rates as a function of flow rate; and ii. the sphere diameter ranging from 0.5-100 μm which defines the penetration of the sphere into the peak flow velocity regions of the substantially laminar flow profile and which defines the magnitude, spatial distribution and rate of change of shear gradients. FIGS. 6b to 6d depict gross variations of the sphere geometry which may arise in alternative embodiments of the present invention. These 3-dimensional geometries or features could range from hemispheres such as 624 shown in FIG. 6(b), tear drop shapes such as 634 shown in FIG. 6(c) which more closely resembles an in situ thrombus shape, and/or convex shapes with varying degrees of camber such as 644 shown in FIG. 6(d).

FIGS. 7a to 7d are schematic views of a polydimethylsiloxane (PDMS) block 700 within which a micro-shear gradient device having sphere geometry micro-channel has been fabricated in accordance with another embodiment of the present invention. FIGS. 7a and 7b give cross sectional views of the microchannel block 700 showing the position and dimensions of the rectangular microchannel 710. FIG. 7c is a top view of the microchannel device 700 with sphere geometry showing the inlet port 720 of diameter 16 mm and outlet port 722 of diameter 2 mm. FIGS. 7d and 7e give detailed side and top view schematics, respectively, of the sphere geometry 714 showing the position of the sphere geometry 714 relative to the microchannel 712. As shown in FIG. 7d, the microchannel 712 is of height 100-200 micrometres, the sphere 714 leaves an overhead gap of width between 50 and 99.75 micrometres, and the outflow channel 718 is of a height in the range of 100-200 micrometres. The sphere 714 is of a diameter between 0.5 and 100 micrometres. As illustrated in the top view of FIG. 7e, the sphere 714 is centrally located on the floor of the channel 712, leaving equal sized side gaps in the range of 50-99.75 micrometres. The inflow channel 716 upstream of microchannel 712 is of width 725 micrometres.

FIG. 8 illustrates an embodiment of the invention in which the protrusion comprises a post 814 in the channel 812. Arrow 810 indicates that a blood sample is perfused left to right through the micro-shear gradient chamber 812. Interaction of the sample with the microscale post geometry 814 leads to lateral shear acceleration immediately upstream and about the post 814, followed by a tightly coupled deceleration phase about and immediately downstream of the post 814, which drives the aggregation of discoid blood platelets at the downstream face of the post geometry 814. Such post geometries are defined by 3 principal parameters: i. the channel width (100-200 μm) which defines and constrains initial blood shear rates as a function of flow rate; ii. the post height ranging from 0.5-100 μm which defines the penetration of the post into the peak flow velocity regions of the substantially laminar flow profile; and iii. the post diameter ranging from 0.5-100 μm that defines the magnitude, spatial distribution and rate of change of shear gradients.

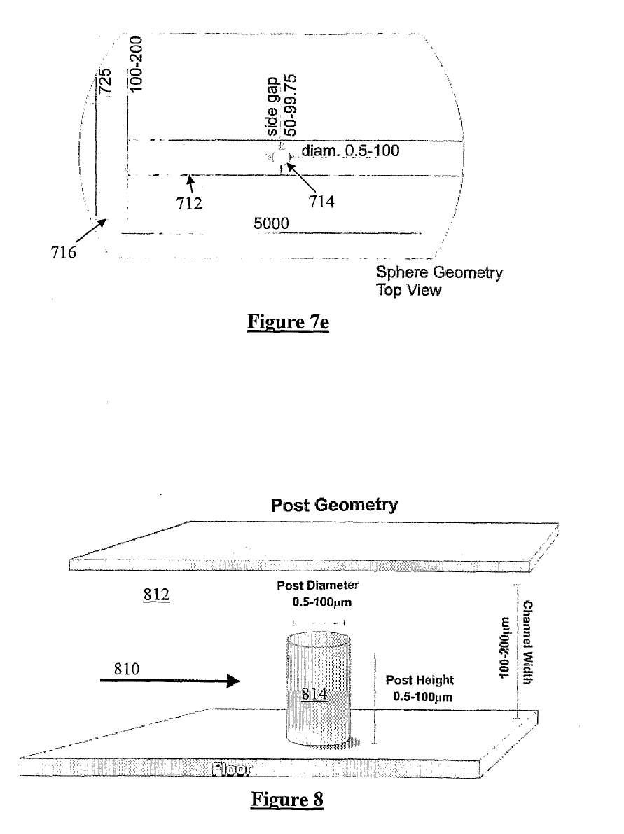

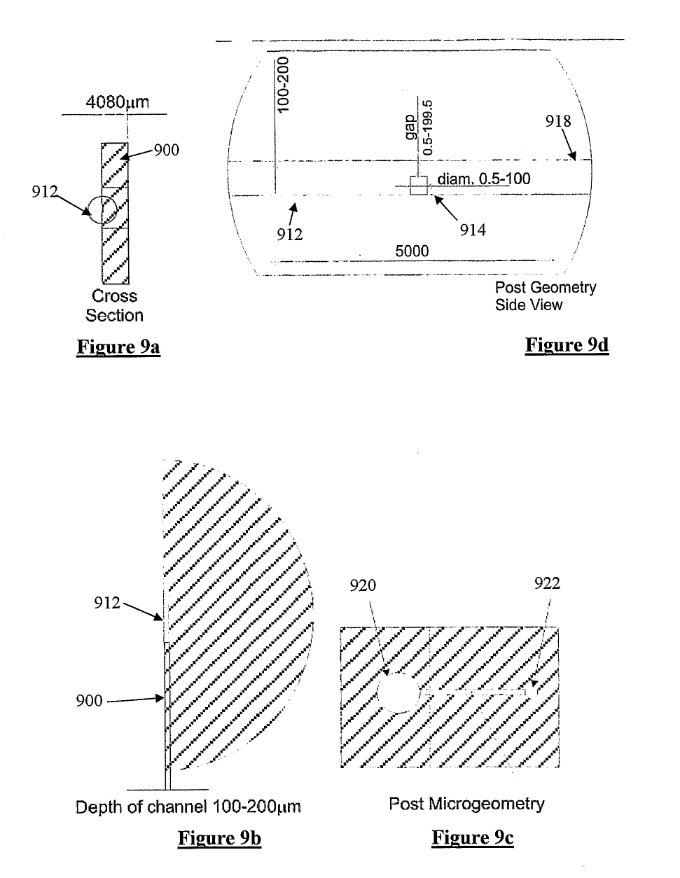

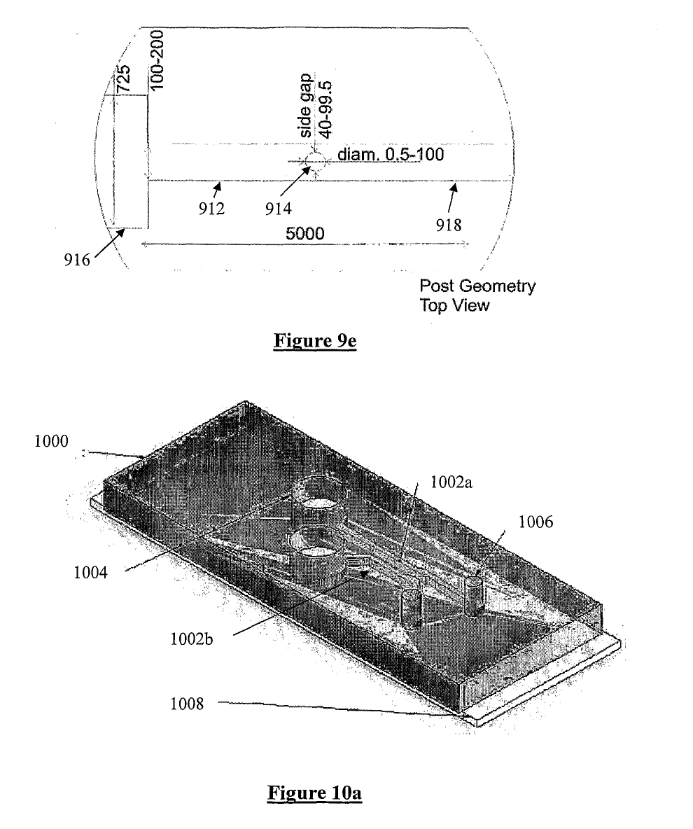

FIGS. 9a to 9e are cross-sectional schematics of a micro-shear gradient device 900 having a post geometry in accordance with a further embodiment of the present invention. FIGS. 9a and 9b are cross sectional views of a microchannel block 900 showing the position and dimensions of the rectangular microchannel 912. FIG. 9c is a top view of the microchannel device 900 with post geometry showing the inlet port 920 of diameter 16 mm and outlet port 922 of diameter 2 mm. FIGS. 9d and 9e are detailed side and top view schematics, respectively, of the post geometry showing position of the post 914 relative to the microchannel 912. As shown in FIG. 9d, the microchannel 912 is of a height in the range 100-200 micrometres, the post 914 leaves an overhead gap of between 50 and 99.75 micrometres, and the outflow channel 918 is of a height in the range of 100-200 micrometres. The post 914 is of a diameter between 0.5 and 100 micrometres, and of a height between 0.5 and 100 micrometres. As illustrated in the top view of FIG. 9e, the post 914 is centrally located on the floor of the channel 912, leaving equal sized side gaps in the range of 50-99.75 micrometres. The inflow channel 916 upstream of microchannel 912 is of width 725 micrometres.

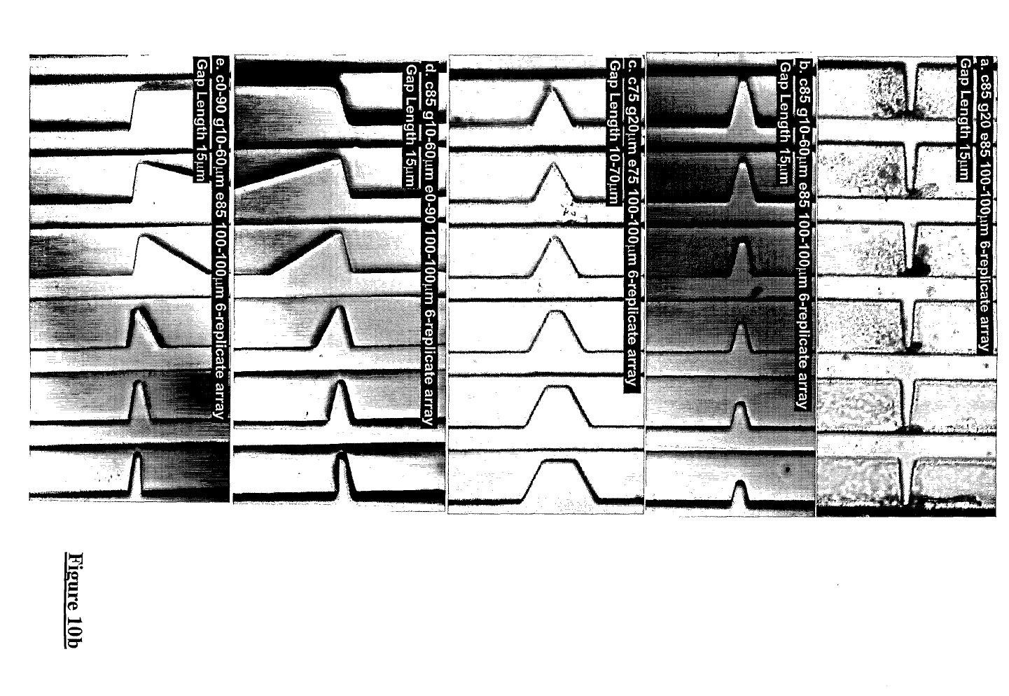

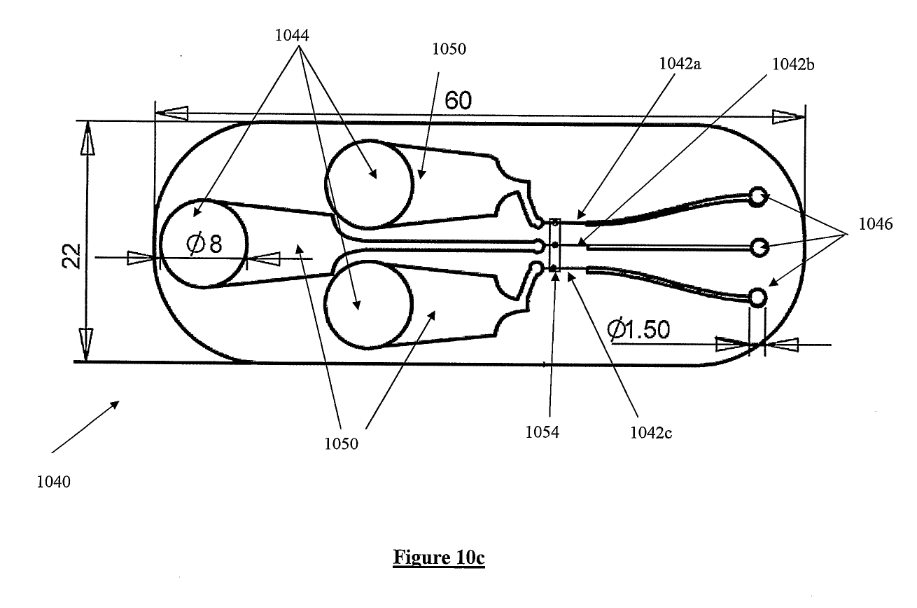

FIG. 10a schematically illustrates a microfluidic device according to an embodiment of the invention. The microfluidic device is in the form of a disposable cartridge which comprises three layers, a first outer layer (not shown), a second outer layer 1008 and the fabricated interposed layer 1000. The cartridge is positionable within a multi-use housing (not shown).

The fabricated interposed layer 1000 has two micro-fabricated flow channels 1002a and 1002b, which apart from unique inlet and outlet geometries, are identical. The microchannels 1002a and 1002b are formed within a Polydimethylsiloxane (PDMS) block which rests on a coverslip 1008 which seals the respective microchannels. At each end of each microchannel 1002a, 1002b is an inlet 1004, and an outlet 1006.

Each channel 1002a, 1002b consists of a five mm long channel of rectangular cross-section, at the centre of which is introduced an asymmetric step or protrusion. The step geometries are defined by six parameters namely:
i) the in-flow channel width (100-1000 μm) which defines and constrains initial blood shear rates to the physiological range (150-10,000 s⁻¹);
ii) the in-flow, or contraction angle (θ_{c}) ranging from 0° through 90° (though more preferably 30° through 90°) that defines the rate of blood flow acceleration;
iii) the step gap height g ranging from 10 μm-40 μm which defines the peak shear following the acceleration phase;

iv) the expansion angle (θ_{c}) ranging from 0° through 90° (though more preferably 30° through 90°) that defines the critical rate of blood flow deceleration into the expansion geometry, defining the zone of platelet aggregation;
v) the expansion channel width ranging from 100 μm-1000 μm that defines the magnitude of the deceleration phase; and
(vi) the gap width ranging from 0.5-20 μm which defines the width of the protrusion or barrier.

### Microchannel Fabrication

The micro channels 1002a, 1002b were fabricated in PDMS, Sylagard from a KMPR 1025 photoresist (microChem Corp) mould, using standard soft-lithography techniques on a 3 inch silicon wafer (Weibel, D. B., Diluzio, W. R. & Whitesides, G. M. Microfabrication meets microbiology. Nature reviews 5, 209-218 (2007)). A high-resolution chrome mask was employed to attain well-defined features to construct the mould. A four inch silicon wafer was spin coated with KMPR 1025 (MicroChem Corp.) photo-resist using a spread cycle of 300 rpm and 100 rpm s⁻¹ for ten seconds and a development cycle of 1000 rpm s⁻¹ and 300 rpm for thirty seconds in order to achieve a film of 130 μm thickness with good uniformity. A cycle of edge bead removal was conducted for thirty seconds using edge bead removal solvent. The KMPR coated wafter was soft-baked by ramping the temperature at 6° C. min⁻¹ starting from 23° C. and holding the temperature at 100° C. for four minutes to dry out the solvents. The KMPR film was exposed with a mask pattern for two minutes of UV on an MJB3 contact mask aligner with a wavelength of 360 nm and a power of 8 mW cm⁻² using two exposures of one minute each in order to avoid over heating the substrate. After exposure the patterns were cross-linked by baking on a hotplate of four minutes at 100° C., ramping the temperature at 6° C. min⁻¹ starting from 23° C. The exposed and cross-linked film was cooled down slowly to room temperature with the sample on the hotplate to avoid thermal stress on the film and possible cracks due to sudden changes in temperature. The exposed KMPR was developed for 12 minutes with periodic agitation to remove the unexposed material. After developing the KMPR pattern, the wafer was cleaned with isopropanol and DI water and a final hard bake was done by heating the sample to 120° C. for three hours, in order to improve and strengthen the cross-linked KMPRO pattern.

The KMPR pattern was then ready for use as a mould for casting PDMS channels. Once the mould was fabricated, PDMS and its curing agent were mixed at a ration of 10:1 and degassed for thirty minutes. The mixture was poured onto the KMPR mould previously made and contained within a ploy methyl methacryalte (PMMA) him. The PDMS was then cured in an oven at 100° C. for twenty minutes. The PDMS channels were peeled from the KMPR mould and 6 mm inlet reservoir holes 1004 were made using biopsy punch. For the outlet connection to the syringe, pump, a 2 mm biopsy punch was used. After both holes were punched, the PDMS channel was placed directly on to a 65×22 mm glass slide 1004. Adhesion was achieved due to the low surface energy of the PDMS.

The first outer layer comprises a 6 mm thick PDMS elongate plate, machined to match the dimensions of the interposed layer 1000. The first outer layer provides a sample inlet which comprises a sample inlet passage and a sample inlet port. The sample inlet passage passes through the first outer layer. The sample inlet port is defined by the sample inlet passage in the outer surface of the first outer layer. The sample inlet passage is machined through the first outer layer and tapped to incorporate M5 fittings to allow quick connection of the cartridge to fluid delivery systems. The first outer layer further provides an outlet which comprises an outlet passage and an outlet port.

The cartridge is assembled by pressing the first layer onto the interposed layer 1000 and adhering one to the other with a pressure-sensitive adhesive. The cartridge is oriented such that the first outer layer forms a top layer and the coverslip 1004 forms a base layer of the cartridge. As assembled, the sample inlet passages of the first outer layer are respectively aligned with the inlets 1004 of the interposed layer. Similarly, the sample outlet passages of the first outer layer are respectively aligned with the outlets 1006 of the interposed layer. The cartridge thus formed defines flow channels 1002a and 1002b The flow channels thus formed run a substantially straight course, and are respectively connected at a first end to the sample inlet 1004 and at a second end to the outlet 1006.

In use of the device, a blood sample or cell suspension from a subject is introduced into the device via the respective inlets and is then perfused through the microchannels 1002a, 1002b at a predetermined flow rate, under the control of a syringe pump, gravity feed, peristaltic pump or any form of pressure driven pump. Platelet aggregation within the microchannels 1002a, 1002b is examined by a detection means, such as DIC, epifluorescence microscopy or other optical method. Notably, as the microchannels 1002a and 1002b are identically configured and the platelet aggregation zones are immediately adjacent, the sum of platelet aggregation within each microchannel 1002a and 1002b can be optically monitored (noting that PDMS is optically transparent). Such a cumulative monitoring method improves reliability of platelet aggregation measurement and reduces the effects of random variations of platelet aggregation within any one microchannel.

Whilst the example here is shown with only two microchannels, a greater number of microchannels may suitably be provided within the PDMS block (for instance 3, 4, 5, 6 or more) in order for instance, to further smoothen such measurements.

FIG. 10b illustrates differential interference contrast images (10× magnification) are illustrated showing several physical embodiments of the device design in a parallel array configuration. The nomenclature cXgYeZ is used, where cX is the angle of the upstream face of the protrusion, gY is the length in micrometers of the gap, and eZ is the angle of the downstream face of the protrusion. Six replicates are demonstrated however the array could be composed of up to 300 different iterations or 300 identical channels each with independent flow (pump) control.

FIG. 10c illustrates a schematic of an alternative device 1040 in accordance with an embodiment of the invention. In contrast to the device 1000 illustrated in FIG. 10a, the step geometry configuration of the micro-shear gradient device 1040 comprises three micro-fabricated flow channels 1042a, 1042b and 1042c, each having unique inlet reservoirs 1044 and outlet reservoirs 1046. The geometries of the respective inlet reservoirs 1044 each have a diameter of 8 mm and each have the same defined strain rate micro-gradient geometry. The geometries of the respective outlet reservoirs 1046 each have a diameter of 1.5 mm.

An upstream trap 1050 is provided in respective of each of the micro-channels 1042a, 1042b and 1042c to at least prevent fouling thereof by particulate matter and/or micro-clots that may have formed in the blood sample due to inadequate anticoagulation. The traps 1050 assist in maximising flow efficiency of blood through the device. A feeder channel 1052 is provided which connects each trap 1050 with the respective microchannel via a single micro-contraction 1054.

In use of the device as shown in FIG. 10a, a blood sample or cell suspension from a subject is introduced into the device via the inlet 1020 and is then perfused through the microchannels 1012 at a predetermined flow rate, under the control of a syringe pump, gravity feed, peristaltic pump or any form of pressure driven pump. Platelet aggregation within the microchannels 1012 is examined by DIC, epifluorescence microscopy or other optical method. Notably, as the microchannels 1012 are identically configured and the platelet aggregation zones are immediately adjacent, the sum of platelet aggregation within each microchannel 12 can be optically monitored (noting that PDMS is optically transparent). Such a cumulative monitoring method improves reliability of platelet aggregation measurement and reduces the effects of random variations of platelet aggregation within any one microchannel. A greater number of microchannels may suitably be provided within the PDMS block 1000 in order to further smoothen such measurements.

The step configuration constitutes a multitude of microscale geometries in which all or one of these parameters has been modified. The key constraint is the overall dimensions of the step such that the length and height scales are in the order of 0-100 μm. In the case of a 0 μm height step or expansion geometry the channel consists of a 100 μm wide channel that expands with a given expansion angle into a straight channel of 200-1000 μm width.

These embodiments of the invention have been developed in recognition that, given the critical role for platelet adhesion processes in haemostasis and thrombosis, there is an important clinical need for the development of a relatively simple and reliable diagnostic test that can accurately assess the adhesive function of platelets in vitro. The traditional uses of platelet function tests have been to identify platelet defects that contribute to a bleeding disorder, monitoring haemostatic therapy in patients at increased risk of bleeding and to ensure normal platelet function in the peri-operative period. However, the above embodiments have been developed under the recognition that simpler and more reliable platelet function tests are potentially also useful for monitoring the effectiveness of antiplatelet therapies, to identify patients with hyper-reactive platelets and increased risk of thrombosis, for quality control of platelet concentrates, for the screening of platelet donors and potentially for the prediction of surgical bleeding risk. The preceding embodiments of the invention have been developed under the recognition that the ideal in vitro platelet function test should be simple to perform, provide rapid and easily interpretable test results, use a small volume of blood (either native or anti-coagulated), be capable of assessing platelet function over a broad range of blood flow conditions, be able to assess both platelet adhesion and aggregation on physiologically-relevant thrombogenic surfaces and be highly reproducible and reliable.

The above embodiments of the invention thus provide devices, and methods incorporating the use of the devices, for assessing platelet aggregation in a biological sample obtained from a subject. The embodiments exploit the recognition that micro-changes in blood flow (shear gradients) represent a general feature of thrombus development in vivo. By providing devices for use ex vivo which include one or more microcapillaries or microchannels, each of which has a defined geometry, these embodiments provide for close mimicking of the natural environment in vivo by mimicking a range of conditions such as flow rate and wall shear stress typical of those which occur in vivo. Such devices therefore have applications for the assessment of thrombus development (or clotting activity) in a subject who may be suspected of having an abnormality in platelet activity or function, such as those occurring in thrombosis, heart disease, stroke or other vascular diseases (including deep vein thrombosis (DVT)), or who may be demonstrating a lack of responsiveness to standard therapy used in the treatment of such diseases (e.g. heparin or other thrombolytic agents), for example.

### Biological Sample

The term “biological sample” as used herein is intended to include any sample containing platelets, including, but not limited to, processed and unprocessed biological samples such as whole blood (native or anticoagulated), plasma, platelets, or red blood cells. In a preferred embodiment, the sample comprises platelets or progenitors thereof.

Without wishing to be bound by theory, the withdrawal of the biological sample using syringe devices can result in shearing of the blood sample. Accordingly, in order to obtain accurate results it is recommended that samples be obtained in a way that minimises shearing. One example is to use a higher guage needle such as a 16 Guage needle for withdrawing the blood sample. Other mechanisms for minimising shearing will be familiar to persons skilled in the art.

The biological sample of the invention is preferably derived from humans or primates. The biological sample may also be derived from a livestock or companion animal.

### Subjects

The term “subject” as used herein is intended to include a healthy subject as well as a subject with known or suspected abnormality in platelet activity or function. The subject includes any of those described above. Preferably the subject is a human subject.

Subjects according to the invention include those with suspected or known bleeding risk, for example Von Willebrand disease subjects, subjects with Bernard-Soulier syndrome, subjects with Glanzmann thrombasthenia, subject with vitamin K deficiency.

Other suitable subject according to the present invention are those with suspected or known clotting risk, for example stroke victims, subjects with diabetes, smokers, subjects with heart disease, subjects who have recently undergone surgery or subjects about to undergo a medical or dental procedure who may be at risk of excessive bleeding.

The term “flow rate” is also referred to throughout the specification by the equivalent ter: “perfusion rate”. The biological sample may be passed unidirectionally through the microcapillary or microchannel using any flow regulating means, such as a single speed pump, a variable speed pump, a syringe pump or gravitational forces. Regulation of the flow rate may be achieved by any suitable method, such as variation in pump speed.

Flow rate is defined as millilitres of fluid per minute. Shear is a consequence of the relative parallel motion of fluid planes during flow, such that in a vessel, the velocity of fluid near the wall is lower than towards the centre. This difference in flow rate between concentric layers of fluid creates a “shearing” effect. Shear is defined as either shear rate or shear stress. Shear rate is expressed as cm/s per cm (or inverse second-s⁻¹). Shear stress is force per unit area (expressed as Dyn/cm² or Pascals) and is equivalent to shear rate×viscosity.

The term “shear micro-gradient” as used in the context of the present invention is intended to refer to the shearing effect caused by a change in velocity of the flow of the biological material. By specifically engineering the microcapillaries or microchannels to have varying inflow and outflow geometries, the present embodiments provide for examination of the effect of differences in shear micro-gradients on platelet aggregation.

Notably, below a flow rate of about 250 microlitres per minute applied to the embodiment of FIG. 10, the Reynolds numbers of the fluid flowing through the microchannel are less than about 26. In this regime the flow rate of blood is stable, without flow separation or vortex formation. In particularly preferred embodiments of the invention the flow rate is about 8 microlitres per minute and the Reynolds numbers are about 0.86, yielding absolutely no opportunity for separation or vortex formation, in contrast to other devices which rely on causing flow separation and vortex formation. Embodiments of the present invention instead exploit decelerating flows and the resulting shear gradients.

More particularly, the present embodiments have been developed in recognition that local shear micro-gradients promote platelet aggregation at a zone where shear deceleration occurs immediately following a zone of high shear acceleration. Thus, a zone of shear acceleration followed by a tightly coupled zone of decelerating shear (shear gradient) is a condition conducive to the development of stabilised platelet aggregates.

It will be appreciated that accurate assessment of platelet function will assist the diagnosis the appropriate management of the treatment of subjects. Furthermore, ongoing monitoring of platelet function will also assist in assessing the response of a subject to a particular treatment regimen.

In particular, the method of the present invention is particularly suitable for determining the risk of a subject of developing a blood clot or platelet thrombus. The risk of a subject developing a clot may be determined by making a comparison between different groups of subjects. For example, a comparison may be made of blood samples from normal healthy subjects and blood samples from subjects with a history or increased risk of developing a blood clot, by comparing the platelet aggregation behaviour of the samples across a number of different microcapillary or microchannel geometries over a standardised, specified period of time at a specified flow rate and temperature.

It will also be appreciated that the device and method of the invention can also be used to discriminate between different platelet defects.

It will be further appreciated that the device and method of the invention can be used to assay the effectiveness of particular drugs or substances. For example, the present inventors have found that a different platelet aggregation profile is observed on specific microchannel geometries between integrilin (a common anti-platelet drug) treated samples and normal samples, from human blood.

Clinical conditions contemplated by the method of the present invention include, but are not limited to, full cardiovascular risk assessment in otherwise healthy subjects; assessment of patients who have suffered a thrombotic event; monitoring of the effectiveness of prescribed anti-platelet therapy; assessment of bleeding or clotting risk in patients scheduled for major surgery; assessment of the clotting risk profile in patients at high risk of cardiovascular disease, including those with diabetes mellitus, hypertension, high blood cholesterol, strong family history of clotting, smokers and those with identifiable thrombosis markers; assessment of clotting risk in patients with peripheral vascular disease; and investigation of the profile of patients with bleeding disorders.

### Reagents

The reagent according to the invention may be a drug or other non-medical substance. For example, the reagent may be selected from anti-platelet drugs, anticoagulants, thrombolytic drugs/fibrinolytics or non-medical such as citrate, EDTA or oxalate.

Examples of suitable anti-platelet drugs include glycoprotein IIb/IIIa inhibitors such as abciximab, eptifibatide and tirofiban; ADP receptor/P2Y₁₂ inhibitors such as thienopyridines (clopidogrel, prasugrel, ticlopidine) and ticagrelor; prostaglandin analogues such as beraprost, prostacyclin, iloprost, treprostinil, COX inhibitors such as acetylsalicyclic acid/asprin, aloxiprin, carbasalate calcium, and others such as ditazole, cloricromen, dipyridamole, indobufen, picotamide and triflusal; vitamin K antagonists such as coumarins: acenocoumarol, coumatetralyl, dicoumarol, ethyl biscoumacetate, phenprocoumon, and warfarin, 1,3-Indandiones: clorindione, diphenadione and phenindione, and others such as tioclomarol.

Examples of suitable anticoagulants include Factor Xa inhibitors such as heparins: bemiparin, certoparin, dalteparin, enoxaparin, nadroparin, parnaparin, reviparin, tinzaparin; oligosaccharides such as fondaparinux, and idraparinux; xabans such as apixaban, otamixaban, and rivaroxaban.

Other suitable anticoagulates include direct thrombin inhibitors such as hirudin (bivalirudin, lepirudin, desirudin), argatroban, dabigatran, melagatran, ximelagatran and others such as REGI, defibrotide, ramatroban, antithrombin III, protein C.

Examples of suitable thrombolytic drugs/fibrinolytics include TPA (alteplase, reteplase, tenecteplase), UPA (urokinase, saruplase), streptokinase, anistreplase, monteplase, and serine endopeptidases such as ancrod and fibrinolysin.

As used herein, the term “reagent” is used in its broadest sense to encompass a single compound or mixture of compounds. The term includes synthetic or natural substances; including biological materials such as antibodies, hormones, other proteins or polypeptides and the like.

The reagent may be an agent that activates platelets such, for example, collagen, ADP, thrombin, thromboxane A₂, serotonin and epinephrine.

The reagent may be, for example, a known anti-platelet agent. Alternatively, the substance may be a substance which is to be screened for its modulating effect on platelets or progenitors thereof, or other cells.

The term “modulation” is used herein to refer to any effect which the substance has on the platelet aggregation activity of platelets or progenitors present within the biological sample. Accordingly, the term encompasses enhancement or inhibition of platelet aggregation activity.

Notably, the present system provides for a shear micro gradient on a downstream side of the protrusion in the zone of platelet aggregation and hence covers a wide range of shear rates, more appropriately mimicking the natural in vivo environment. Further, the present system does not require the manipulation of blood samples prior to assay. Still further, the present system can be used with small blood volumes. This is particularly important in the paediatric setting where blood volumes harvested from babies or toddlers are smaller and/or difficult to obtain. Still further, the present system does not rely on rates of occlusion but rather allows platelet aggregation to proceed to dynamic equilibrium and therefore gives information on maximal thrombus size. Still further, the present system allows for the measurement of thrombus stability in real-time.

A further advantage of some embodiments of the present system is that the present device permits the visualisation and analysis of platelet aggregation to be monitored in real-time. Still further, the present system is capable of giving kinetic data on platelet aggregation rate and extent.

It will be appreciated by persons skilled in the art that numerous variations and/or modifications may be made to the invention as shown in the specific embodiments without departing from the scope of the invention as broadly described. The present embodiments are, therefore, to be considered in all respects as illustrative and not restrictive.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Further features of the present invention are more fully described in the following detailed description and examples. It is to be understood, however, that the examples are included solely for the purposes of exemplifying the present invention. It should not be understood in any way as a restriction on the broad description of the invention as set out above.

### EXAMPLES

### Methods

### Collection of Blood Samples

Blood samples from subjects were taken via venisection of the antecubital vein using a 10 ml syringe fitted with a 19 guage needle containing 800 U/ml hirudin as anticoagulant.

### Treatment of Blood Samples

Blood perfusion studies through the device were carried out using hiridin (800 U/ml) anticoagulated whole blood taken from consenting human donors. Whole blood samples were incubated at 37° C. for 10 minutes with the lipophylic membrane dye DiOC₆ (1 μg/ml) or DiICl2 (1 μg/ml) to allow the cells to be more readily visualised through the device.

### Microchannel Fabrication

Micro channels were fabricated in polydimethylsiloxane (PDMS, Sylagard) from a KMPR 1025 photoresist (microChem Corp) mould, using standard soft-lithography techniques on a 3 inch silicon wafer (Weibel, D. B., Diluzio, W. R. & Whitesides, G. M. Microfabrication meets microbiology. Nature reviews 5, 209-218 (2007)). A high-resolution chrome mask was employed to attain well-defined features to construct the mould. The KMPR was developed using Su8 developer. This formed an inverse mould of the channels. The overall channel depth was 80 μm. Having constructed the mould, Polydimethylsiloxane (PDMS) and curing agent were mixed with a ratio of 10 to 1 and degassed for 20 minutes and poured on the mould to a thickness of approximately 4 mm. The PDMS was cured for 20 minutes at 90° C. Finally the PDMS channel was bonded directly onto a borosilicate cover glass of 160 μm of thickness.

To achieve the tolerances needed, a high degree of quality control is required at each stage of the photolithographic process. As an example of this, sharply defined corners were part of the preliminary design however due to current limitations in the fabrication process; rounded corners were produced on the order of a 5 micrometer radius (which was a consideration in the CFD modeling). Although this limitation did not grossly impact on the platelet response, further refinement of the fabrication method may ultimately result in more precise control of the hemodynamics and resultant platelet aggregation. Although PDMS offers many advantages in terms of cost and ease of fabrication, other materials that allow for more precise geometries may prove to be advantageous in other embodiments of the present invention.

### Numerical Simulations of Fluid Flow (CFD)

The estimation of the strain rates were calculated after solving the velocity field using the conservation of mass and momentum equations for an incompressible fluid flow, using the computational fluid dynamics (CFD) software package FLUENT 6.0 (Fluent USA, Lebanon, N.H.) based on a finite volume scheme, more details of the implementation can be found in the FLUENT manuals. The validity of the continuum hypothesis and the no-slip boundary condition were assumed to hold. The flow is considered as three dimensional, steady, laminar and incompressible. The fluid medium was considered with a constant density of 998.2 km/m³ and viscosity of 0.00345 Pa s. The discretization scheme pressure was standard, and the second order upwind momentum option was enabled in the calculation.

### Example 1

### Blood Perfusion Through a Step Geometry

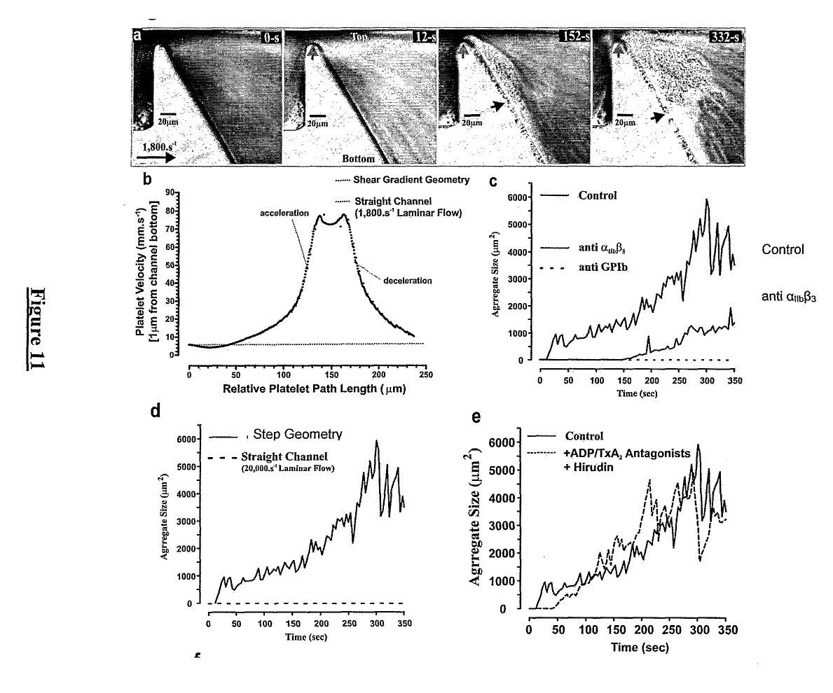

FIG. 11a is a representative micrograph (40× magnification) sequence of human (hirudin anti-coagulated) blood perfusion through a micro-shear gradient device consisting of an in-flow (entry) width of 100 μm, a contraction angle (θ_{c}) of 90°, a gap height of 10 μm, an expansion angle (θₑ)) of 60°, and an expansion/exit width of 700 μm. The grey arrow denotes the point of initial aggregation [t=12 sec], the black arrow designates the limit of thrombus growth in the expansion zone (representative of n=3 experiments).

FIG. 11b illustrates the results produced by computational fluid dynamics (CFD) simulation (Velocity v displacement plot) showing the velocity change for a platelet (particle) travelling 1 μm (½ discoid platelet diameter) from the surface of the micro-channel wall geometry in (a). In the case of a straight microchannel segment (1,800.s⁻¹ laminar flow), the platelet travels at constant velocity throughout its path length. There is a rapid acceleration phase coupled to a rapid deceleration phase as the platelets travel through the shear gradient geometry.

FIG. 11c comprises representative aggregation traces showing the response of whole blood perfusion through the PDMS microchannel device depicted in FIG. 11a. Step Geometry—represents hirudin-anticoagulated whole blood perfusion at an input (pre-stenosis) shear rate of 1,800.s⁻¹ (representative of n=3 experiments); anti-α_{IIB}β₃-hirudin anticoagulated whole blood treated for 10 minutes with 30 μg/ml c7E3 Fab prior to blood perfusion (representative of n=2 experiments). Note, in the absence of integrin α_{IIB}β₃ engagement, initial recruitment at the stenosis apex is markedly delayed and overall aggregation suppressed; anti-GPIb -,hirudin anticoagulated whole blood treated for 10 minutes with 50 μg/ml of the anti-GPIb blocking IgG ALMA12 (representative of n=3 experiments). Note the complete absence of platelet interaction in the absence of GPIb/V/IX engagement.

FIG. 11d shows representative aggregation traces showing the response of whole blood perfusion through the microchannel depicted in FIG. 11a in comparison with a straight microfluidic device that does not induce a shear gradient; Step Geometry—represents hirudin-anticoagulated whole blood perfusion at an input (pre-stenosis) shear rate of 1,800.s⁻¹ (representative of n=3 experiments); Straight Channel—hirudin-anticoagulated whole blood perfusion through a 100 μm straight microchannel at a bulk shear rate of 20,000.s⁻¹ (representative of n=3 experiments);

FIG. 11e comprises representative aggregation traces showing the soluble agonist independence of the platelet aggregation response of whole blood perfusion through the micro-shear gradient device depicted in (a); Step Geometry—represents hirudin-anticoagulated whole blood perfusion at an input (pre-stenosis) shear rate of 1,800.s⁻¹; +ADP/TXA₂ Antagonists+Hirudin hirudin anticoagulated whole blood treated for 10 minutes with MRS2179 (100 μM), 2-MeSAMP (10 μM) and Indomethacin (10 μM) (representative of n=3 experiments).

Trial blood flow experiments using hirudin (50 mg/kg) anticoagulated whole blood in a sample of the proposed step geometries have demonstrated that as per the shear gradient model of platelet aggregation, platelet thrombi form exclusively within the identified flow deceleration zone at the downstream face of the step geometries (see FIG. 11a). Control studies have demonstrated that sustained, elevated laminar shear (20,000.s−1) in a straight microchannel without a step geometry are incapable of inducing the platelet proaggregatory phenotype (FIG. 11b-d). In accordance with the shear gradient model of platelet aggregation, blockade of chemical platelet agonists (ADP and TXA2) does not block shear gradient dependent aggregation at step geometries (FIG. 11e). However this aggregation process is critically dependent on platelet integrin αIIbβ3 engagement (FIG. 11c).

### Example 2

### Flow Rate Dependency of Two Step Geometry Iterations

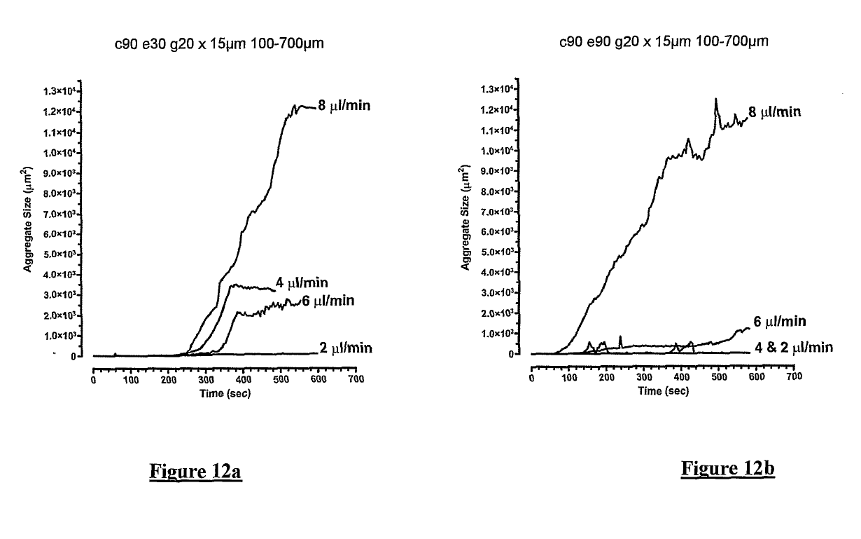

FIG. 12a comprises representative aggregation traces as a function of flow rate (Q=2, 4, 6 & 8 μl/min) through a micro-shear gradient device consisting of an in-flow/entry width of 100 μm, a contraction angle (θ_{c}) of 90°, gap height of 20 μm, an expansion angle (θₑ) of 30°, and an expansion/exit width of 700 μm.

FIG. 12b is representative aggregation traces as a function of flow rate (Q=2, 4, 6 & 8 μl/min) through a micro-shear gradient device consisting of an in-flow width of 100 μm, a contraction angle (θ_{c}) of 90°, gap height of 20 μm, an expansion angle (θₑ) of 90°, and an expansion width of 700 μm.

Analysis of flow rate (Q=2, 4, 6 & 8 μl/min) dependency was examined in two step geometry configurations with expansion angles of 30° and 90° (FIGS. 12a and 12b respectively). The size of the aggregates decreased significantly with decreasing flow rate below 8μl/min in the case of both geometries. There was a decreased time to initial aggregation observed for the second geometry (FIG. 12b) having θₑ=90° suggesting that the onset of platelet aggregation for human blood is critically dependent on the expansion angle geometry.

### Example 3

### Sphere Geometry

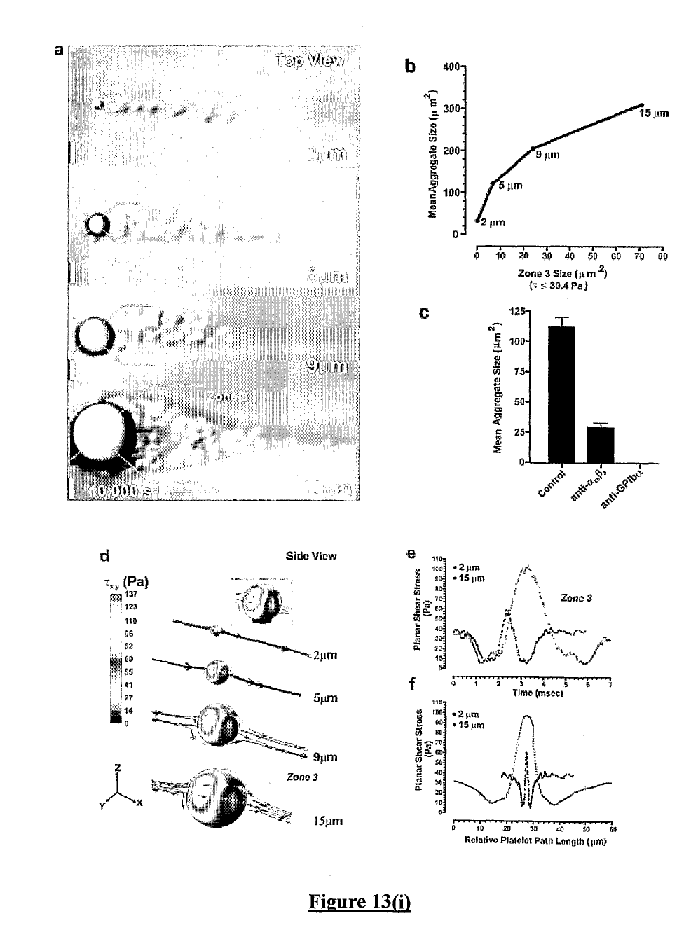

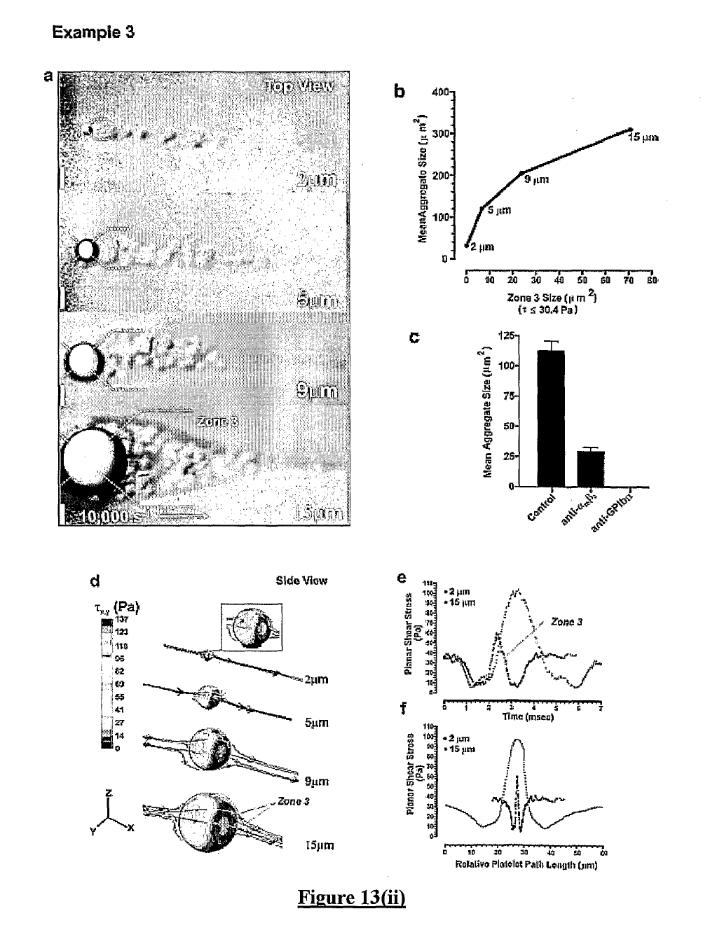

FIG. 13a comprises DIC image frames showing the nature and extent of discoid platelet aggregation at the downstream face of VWF coated sphere geometries following whole human blood (pre-treated with 100 μM MRS2179, 10 μM 2-MeSAMP and 10 μM Indomethacin) perfusion at an applied γ of 10,000.s⁻¹ (n=5).

FIG. 13b illustrates mean discoid platelet aggregate size (surface area in μm²) as a function of the downstream low τ_{x,y} pocket (zone 3 surface area μm²) exhibiting τ≦30.4 Pa (n=3).

FIG. 13c shows mean discoid platelet aggregate size at the downstream face of 5 μm VWF coated sphere geometries at an applied bulk γ of 10,000.s⁻¹; Control—hirudin anticoagulated whole blood; anti-α_{IIb}β₃—hirudin anticoagulated whole blood treated for 10 minutes with 30 μg/ml c7E3 Fab prior to blood perfusion; anti-GPIb—,hirudin anticoagulated whole blood treated for 10 minutes with 50 μg/ml of the anti-GPIb blocking IgG ALMA12 (n=3).

FIG. 13d shows the results of CFD simulation of blood planar shear stresses (τ_{x,y}) around a sphere geometry at an applied bulk γ of 10,000.s⁻¹.

FIGS. 13e & f show the results of CFD analysis of an individual platelet trajectory at a distance of 1 μm (½ platelet diameter) from the lateral surface of a 2 and 15 μm sphere geometry.

Trial blood flow experiments using hirudin anti-coagulated whole blood in a small sample of the proposed sphere geometries have demonstrated that as per the shear gradient model of platelet aggregation, platelet thrombi form exclusively within the identified flow deceleration zone at the downstream face of the sphere geometries (FIG. 13a).

Significantly, the extent of platelet aggregation has been demonstrated to be critically dependent on spherical diameter and input flow rate. As shown in FIG. 13d, there is an increase in shear stress (τ_{x,y}) at the sphere sides as a function of diameter and a low τ_{x,y} zone (zone 3) at the downstream face of the sphere; the size of which is directly dependent on spherical diameter. Planar shear stress (τ_{x,y}) represents the predicted stress experienced by a free flowing platelet perpendicular to the bead surface at a distance equivalent to ½ platelet diameter.

Platelets will experience varying magnitudes and rate of change in τ_{x,y} dependent on their position relative to the sphere surface. Particle path lines at the bead equators experience a τ_{x,y} increase approaching 100 Pa (15 μm beads) that subsequently decreases to less than 30.4 Pa in zone 3. Significantly, the rate of change (τ_{x,y} ν time), spatial distribution (τ_{x,y} ν path length) and peak τ_{x,y} are significantly reduced for the smaller (2 μm) case, however this shear gradient is still capable of inducing a robust aggregation response.

### Example 4

### Platelet Aggregation Dynamics in Three Microchannel Geometries

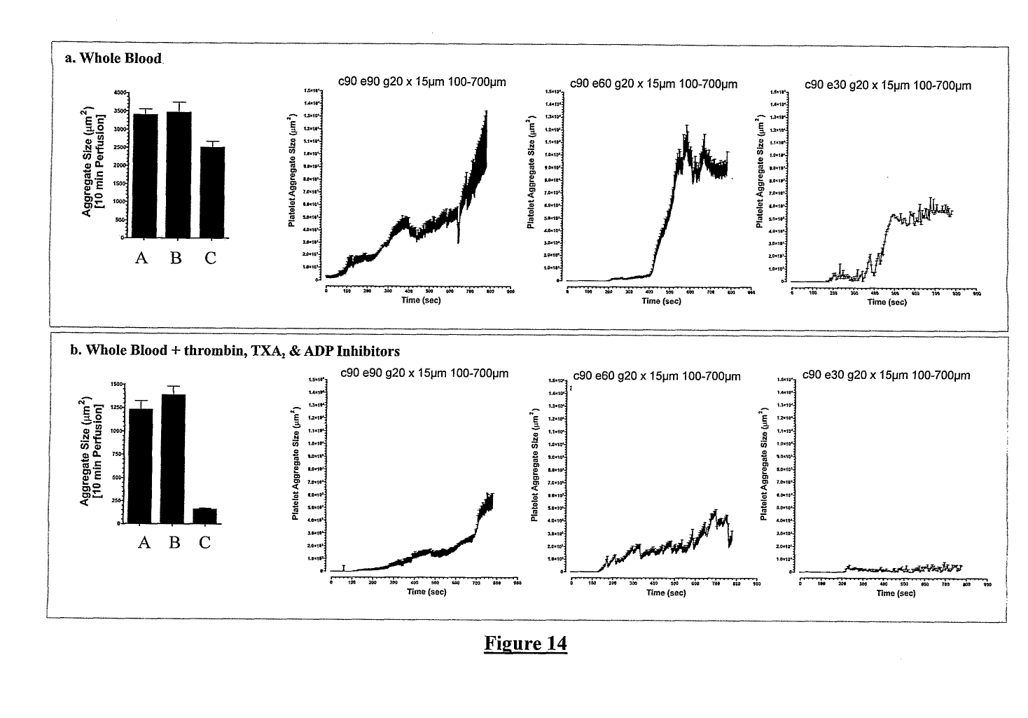

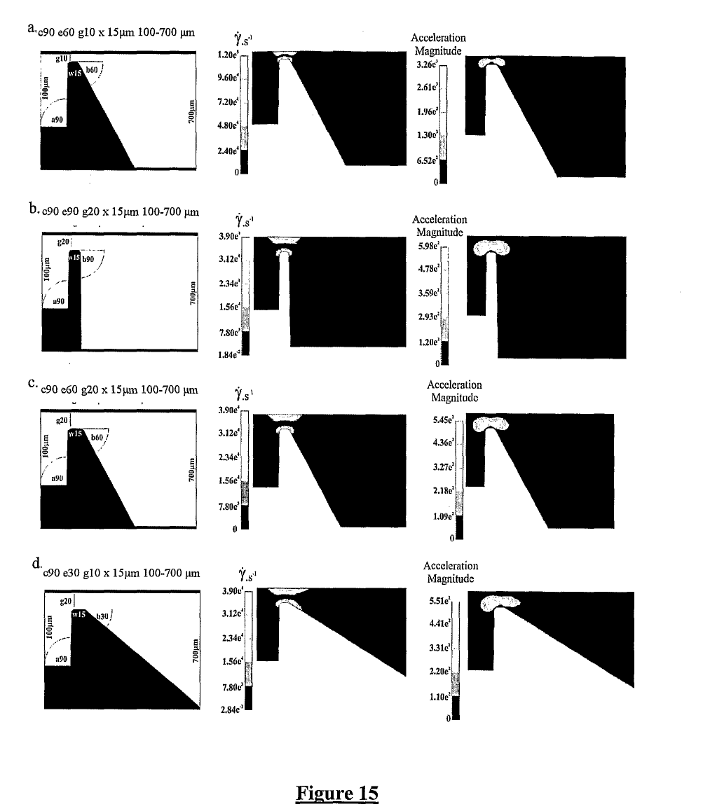

FIG. 14a illustrates results from hirudin-anticoagulated whole human blood perfusion through channel geometries consisting of a 100 micrometre inflow segment, contraction angle of 90° (c90), peak gap of 20×15 micrometres, outflow segment of 700 micrometres and expansion angles varying from 90 to 30° (e90, e60, e30). Mean platelet aggregate size (panel 1) was determined following 10 minutes of whole blood perfusion at an input strain rate of 1,800.s−1 with peak strain at the apex of the micro-geometry approaching 20,000.s−1. Combined data from n=5 independent blood donors (SEM shown). Panels 2-4 show platelet aggregation dynamics at the three specified micro-geometries over a 13 min time frame. Traces are composites derived from n=5 independent blood donors (SEM shown).

FIG. 14b illustrates results from hirudin-anticoagulated whole blood treated for 10 minutes with MRS2179 (100 μM), 2-MeSAMP (10 μM) and Indomethacin (10 μM) to inhibit platelet amplification signalling. This data set shows the direct effect of blood flow parameters on the platelet aggregation response independent of the compounding effects of platelet secretion. Blood samples were perfused through channel geometries consisting of a 100 micrometre inflow segment, contraction angle of 90° (c90), peak gap of 20 micrometres, gap length of 15 micrometres, outflow segment of 700 micrometres and expansion angles varying from 90 to 30° (e90, e60, e30). Mean platelet aggregate size (panel 1) was determined following 10 minutes of whole blood perfusion at an input strain rate of 1,800.s−1 with peak strain at the apex of the micro-geometry approaching 20,000.s−1. Combined data from n=5 independent blood donors (SEM shown). Panels 2-4 show platelet aggregation dynamics at the three specified micro-geometries over a 13 minute time frame. Traces are composites derived from n=5 independent blood donors (SEM shown).

### Example 5

### Acceleration & Strain Rate Analysis of Four Microchannel Geometries

FIG. 15a illustrates a strain rate and acceleration analysis for a step geometry consisting of a 100 micrometre inflow segment, contraction angle of 90° (a90), peak gap of 10×15 micrometres, expansion angle of 60° (e60) and an outflow segment of 700 micrometres. Associated strain rate (γ .s⁻¹) and acceleration magnitude CFD analysis is shown (panels 2 & 3) demonstrating the effect of the micro-geometry on blood flow.

FIG. 15b illustrates strain rate and acceleration analysis for a step geometry consisting of a 100 micrometre inflow segment, contraction angle of 90° (c90), peak gap of 20 micrometres, gap length of 15 micrometres, expansion angle of 90° (e90) and an outflow segment of 700 micrometres. Associated strain rate (γ .s⁻¹) and acceleration magnitude CFD analysis is shown (panels 2 & 3) demonstrating the effect of the micro-geometry on blood flow.

FIG. 15c illustrates strain rate and acceleration analysis for a step geometry consisting of a 100 micrometre inflow segment, contraction angle of 90° (c90), peak gap of 20 micrometres, gap length of 15 micrometres, expansion angle of 60° (e60) and an outflow segment of 700 micrometres. Associated strain rate (γ .s⁻¹) and acceleration magnitude CFD analysis is shown (panels 2 & 3) demonstrating the effect of the micro-geometry on blood flow.

FIG. 15d illustrates strain rate and acceleration analysis for a step geometry consisting of a 100 micrometre inflow segment, contraction angle of 90° (c90), peak gap of 20 micrometres, gap length of 15 micrometres, expansion angle of 30° (e30) and an outflow segment of 700 micrometres. Associated strain rate (γ .s⁻¹) and acceleration magnitude CFD analysis is shown (panels 2 & 3) demonstrating the effect of the micro-geometry on blood flow. This demonstrates the establishment of a strain rate (shear) gradient at the channel geometry at a blood flow rate of 110 min, being the rate required in this particular fabricated geometry to achieve input shear of 1,800 s⁻¹. Note the establishment of three distinct shear zones as defined previously: i. Zone 1—shear acceleration zone; Zone 2—peak shear zone; and Zone 3 shear deceleration or aggregation zone.

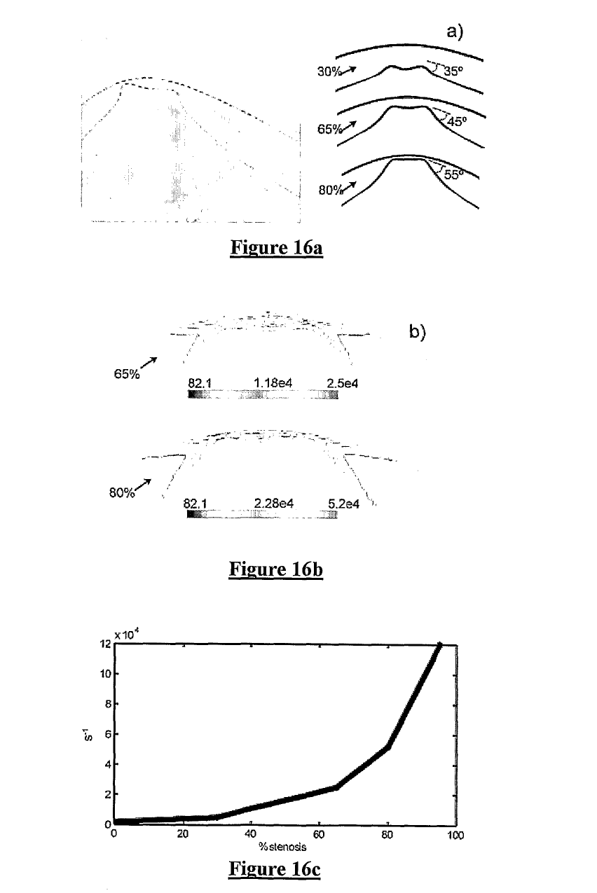

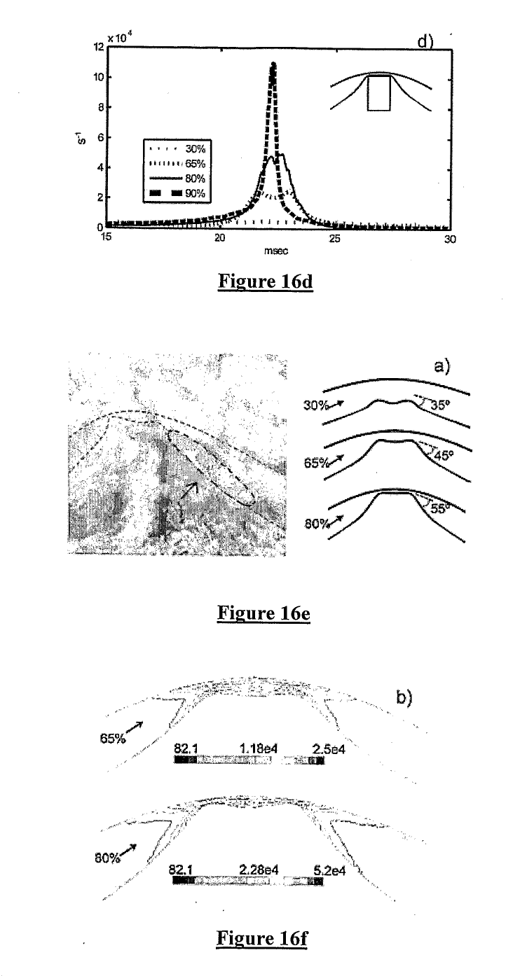

To gain insight into the effects of changing wall geometry on the strain rate environment experienced by platelets under the model conditions, strain rate histories of blood elements within 1 μm (½ platelet diameter) of the vessel wall for four different degrees of stenosis was analysed as illustrated in FIGS. 16a-d.

FIG. 16 shows structural and CFD simulations of a representative mouse mesenteric arteriole undergoing side wall compression. FIG. 16a is a representative micrograph taken from intravital video footage showing stenosis (˜80% of area reduction) of a mouse mesenteric arteriole (42 m in diameter) undergoing vessel side-wall compression with a glass microneedle (dotted line) following crush injury. Platelet aggregate formation is demarcated in yellow shading in FIG. 16a (and depicted by the region indicated by the arrow shown in the corresponding black and white figure of 16e), with the flow direction from left to right. An angle between the main direction of the flow and the wall is produced as the tip of the needle contacts the vessel wall. The associated schematic shows structural model predictions of the effect of progressive vessel side-wall compression at 30, 65 and 80% stenosis. Note that the contraction and expansion angles are predicted to progressively increase from 35-55° as a function of degree stenosis. The black arrow denotes the direction of blood flow.

FIG. 16b shows contour plots of the predicted strain rate distributions for stenoses of 65 and 80%, depicted in FIG. 16a. Note that the reduction of the hydraulic area produces a progressive increase/decrease of the deformation rates of the fluid, going from zones of dark blue (lowest values) to zone of red (highest values). These changes are clearly dependent on the geometry and angles produced by the needle and locally may affect the experienced stress for a particle travelling in the vessel.

FIG. 16c shows the maximum strain-rate at the mouse blood vessel wall as a function of degree stenosis (vessel compression). Note that an exponential relation occurs between the maximum wall strain rate and the degree of stenosis, for a constant flow rate.

FIG. 16d gives predicted (CFD) strain-rate histories for a platelet travelling at 1 micrometer (½ platelet diameter) from the side-wall deformed by microneedle compression for four different degrees of stenosis (30, 65, 80 & 90% of area). Note that an increase in the strain rates is evident as soon as the platelet enters the contraction. A particle travelling in this streamline experiences acceleration, a peak shear zone and a deceleration within a few milliseconds. It was found that for a 65% stensosis (area), a modest increase in strain rate is predicted, while for a degree stenosis of 80% platelets experience a 2-fold increase in strain rate as they pass through the stenosis contraction. Furthermore, the modelling predicts that an increase in 5% (2.1 μm) in severe stenosis (above 80%) results in a 3-fold strain rate increase (40,000-120,000 s⁻¹), suggesting that minor modifications of the vessel side wall at or above 80% stenosis can have a dramatic effect on the strain rate history of platelets flowing through the vessel.

Taken together with the investigators in vivo observations, these numerical simulations of FIG. 16 predict that platelets close to the vessel wall passing through a stenosis experience both rapid and extreme phases of shear acceleration and deceleration with peak strain rates approaching 1×10⁶ s⁻¹. Although these values are extremely high, it has been suggested that platelets are able to withstand elevated shear stresses in the order of 1000 Pa (2.6×10⁵ s⁻¹ strain rate) if the stress duration is within 1-5 milliseconds. This analysis enabled us to identify three principle geometric parameters that may significantly influence platelet function and aggregation within the context of the investigators vascular mimetic design:
i. the contraction angle and associated rate of blood flow acceleration;
ii. the stenosis gap diameter (% stenosis) and associated peak strain rate; and
iii. the expansion angle and associated rate of blood flow deceleration.

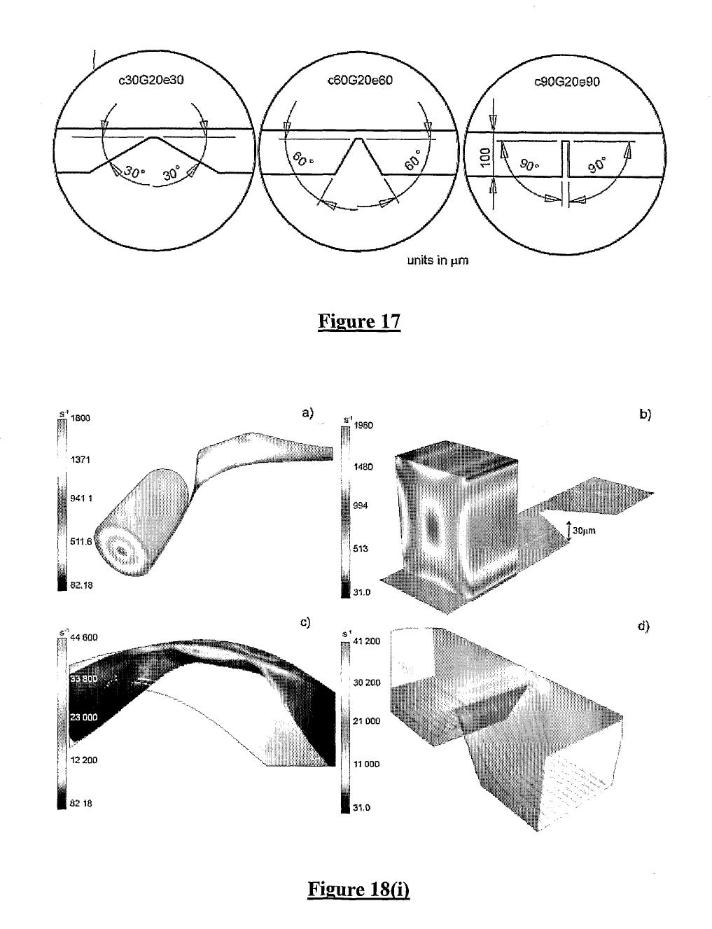

FIG. 17 illustrates the three symmetric micro-channel design cases chosen from all possible cases for the investigators proof-of-concept study. Again, the nomenclature cXgYeZ is used, where eX is the angle of the upstream face of the protrusion, gY is the length in micrometers of the gap, and eZ is the angle of the downstream face of the protrusion. Numerical (CFD) simulations were carried out to predict the velocity field, strain rate distribution produced, and to study particle behavior within selected streamlines of blood flow within the device.

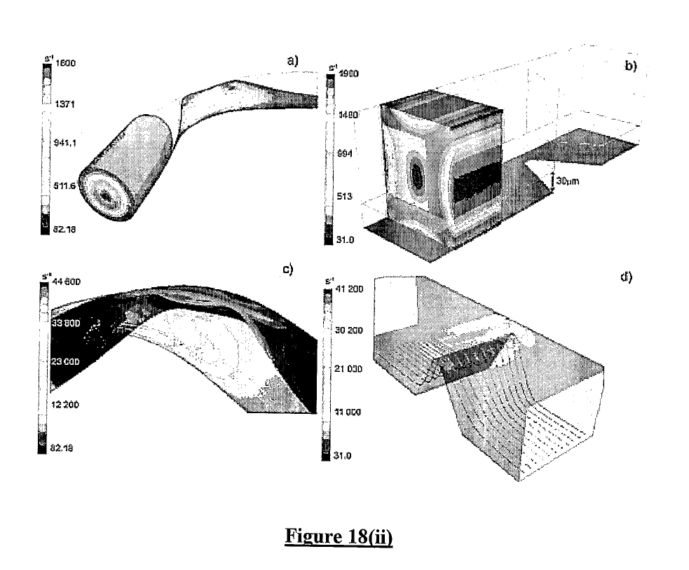

FIGS. 18a to 18d respectively show computed strain rate distributions in the mesenteric arteriole and the c60g20e60 vascular mimetic. FIG. 18a illustrates the computed strain rate distribution colour map for blood flow in the mouse mesenteric arteriole (42 micrometers) upstream of stenosis (side-wall compression). Note, due to viscous effects and the cylindrical geometry, a uniform strain rate at the wall is produced by the fluid flow.

FIG. 18b shows computed strain rate distribution colour map for blood flow in the c60g20e60 vascular mimetic upstream of the defined contraction geometry. Note, due to the rectangular channel geometry and low aspect ratio the flow inside the micro channel produces a parabolic distribution along the walls, with strain rate maxima at the center and minima at the corner edges. A plane located at 30 micrometers from the cover slip was chosen for all imaging experiments such that fluid and particles at this location experience strain rates in the order of ˜1700.s⁻¹, with maxima at the 65 micrometer mid-plane exhibiting strain rates approaching 1960.s⁻¹.

FIG. 18c shows computed strain rate distribution colour map for blood flow in the mouse mesenteric arteriole at a stenosis of 80% area. The geometry of the blood vessel in the contraction zone is imposed by the combination of the shape of the blunted needle and elastic effects of the vessel side-wall. An irregular surface topography is produced which creates a heterogeneous strain rate distribution in 3-dimensions, with two peaks of 44,600.s⁻¹ that rapidly decrease approaching the expansion zone.

FIG. 18d shows computed strain rate distribution colour map for blood flow in the c60g20e60 vascular mimetic at the defined contraction geometry. Note that in the vascular mimetic a bigger aspect ratio is produced resulting in a more homogeneous strain rate distribution. The streamlines shown represent the computed trajectories for particles traveling at 1 micrometer (½ platelet diameter) from the microchannel wall; note that at this distance, a maxima of 41, 200.s⁻¹ is generated, although higher strain rates may be experienced at the wall, where flow velocity is zero.

FIGS. 18a & 18b thus present comparative strain rate distributions (upstream of contraction) for the model blood vessel and the c60g20e60 microchannel format, respectively. Note that in the blood vessel an axisymetric/homogeneous strain rate distribution is predicted, however due to the rectangular geometry and low aspect ratio (width-height=1.3) of the microchannel format, the flow follows a parabolic distribution along the side walls, with the maximum at the center of the walls and the lowest values at the corners (FIG. 18b). However, if the plane of blood flow observation is restricted to the flow volume located between 30-65 micrometers, platelets will experience uniform strain rates ranging between 1,500-1,960 s⁻¹, falling well within the nominal physiological range reported for mesenteric arteries and arterioles. A more homogeneous distribution of strain rates across the channel (across the dimension defined by the photoresist thickness), could be achieved by increasing the aspect ratio of the channel (either increasing the width given by the designed mask or increasing the height, given by the thickness of the photoresist), however, this could affect the hydraulic diameter (affecting the Reynolds number at the contraction and the exposure time of the platelets to the strain rate gradient). In this investigation, the investigators were interested in keeping the lowest possible Reynolds number at the contraction, with a similar residence time, to model a high strain rate zone with similar inertia effects to the in vivo case (Reynolds at the contraction in vivo is 0.45 and in the microchannel Re 2.4).

FIGS. 18c & 18d present the computed results for the strain rate distributions within the contraction zone for the model arteriole (80% stenosis) and c60g20e60 microchannel format, respectively. Note that in the vessel case, the non-uniform nature of the micro-needle compression results in an uneven side-wall topography producing an irregular distribution of strain rate, with two local regions of high shear (˜44,600 s⁻¹) positioned at the upstream and downstream edges of the contraction zone (FIG. 18c). In contrast, a key advantage of the synthetic c60g20e60 microchannel format is that the geometric shape of the contraction is uniform (with a larger aspect ratio) resulting in a more homogeneous strain rate distribution (FIG. 18d). Furthermore, FIG. 18d demonstrates, that for flow streamlines at 1 micrometer from the c60g20e60 microchannel wall, platelets will experience a predicted peak strain rate at the centre of the contraction geometry of 41,200 s⁻¹ that closely approximates the blood vessel. Overall the investigators simulations suggest that the c60g20e60 microchannel format represents a good idealized approximation of the hemodynamic conditions generated within the published in vivo model.

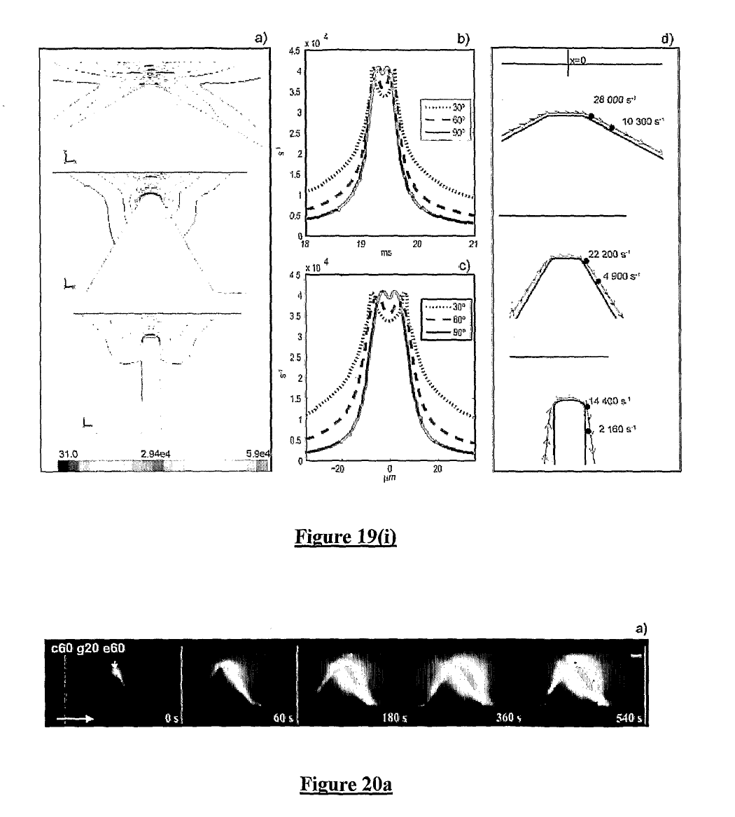

FIGS. 19a to 19d respectively illustrate hydrodynamic performance of the device. FIG. 19a shows contour plots of the predicted strain rate distributions for the c30g20e30, c60g20e60 and c90g20e90 vascular mimetics. Note that the reduction of the hydraulic area produces a progressive increase and then decrease of the deformation rates of the fluid, going from zones of dark blue (lowest values) to zones of red (highest values), however the rate of the “progressive” increase to decrease is different for each geometry.

FIG. 19b shows CFD plots demonstrating the predicted strain rate “history” experienced by a model platelet traveling at 1 micrometer from the step-wall for the three designated microchannel geometries as a function of time.

FIG. 19c shows CFD plots demonstrating the predicted strain rate “history” experienced by a model platelet traveling at 1 micrometer from the step-wall for the three designated microchannel geometries as a function of distance. The 0 micrometer reference point is located at the mid-line of the defined contraction geometries.

FIG. 19d shows CFD plots showing a comparison of the strain rate gradient experienced at 10 micrometers and 30 micrometers from the mid-line of the defined contraction geometries following a streamline 1 micrometer from the step-wall.

FIGS. 19a to 19d thus provide insight to a key hydrodynamic variable that the investigators aimed to modify by changing the expansion angles through 60° in the investigators proof-of-concept geometries, namely the overall deceleration gradient experienced by platelets that initially tether within the contraction zone. FIG. 19c shows an analysis of the effect of expansion angle on the strain rate deceleration experienced by a platelet as it transitions into the expansions for the three microchannel formats. Examination of the instantaneous values of strain rate experienced by a platelet 1 micrometer from the step-wall at 10 micrometers and 30 micrometers from the center of the contraction zone (peak phase) demonstrates that a platelet experiences significant differences in the magnitude of strain rate deceleration as a function of the three angles over an equivalent distance, such that; a θₑ=30° results in a 35% (41,000-28,000 s⁻¹) reduction, a θₑ=60° results in a 46% reduction (41,000-22,200 s⁻¹), and a θ_{c}=90° results in a 65% reduction (41,000-14,400 s⁻¹) in strain rate over the first 10 micrometers of the expansion zone (FIG. 19c).

### Example 6

### Platelet Aggregation as a Function of Microchannel Design

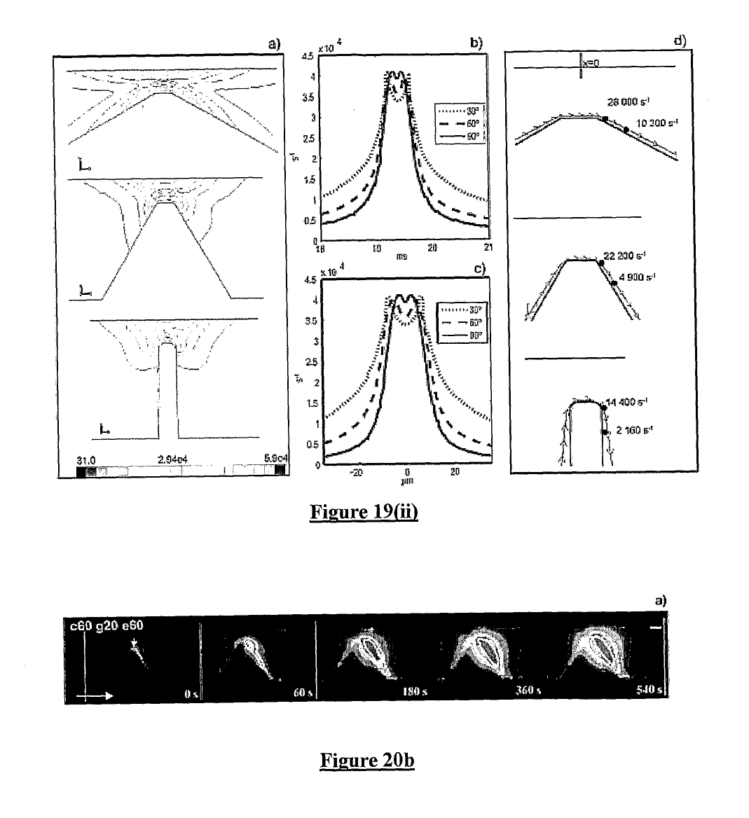

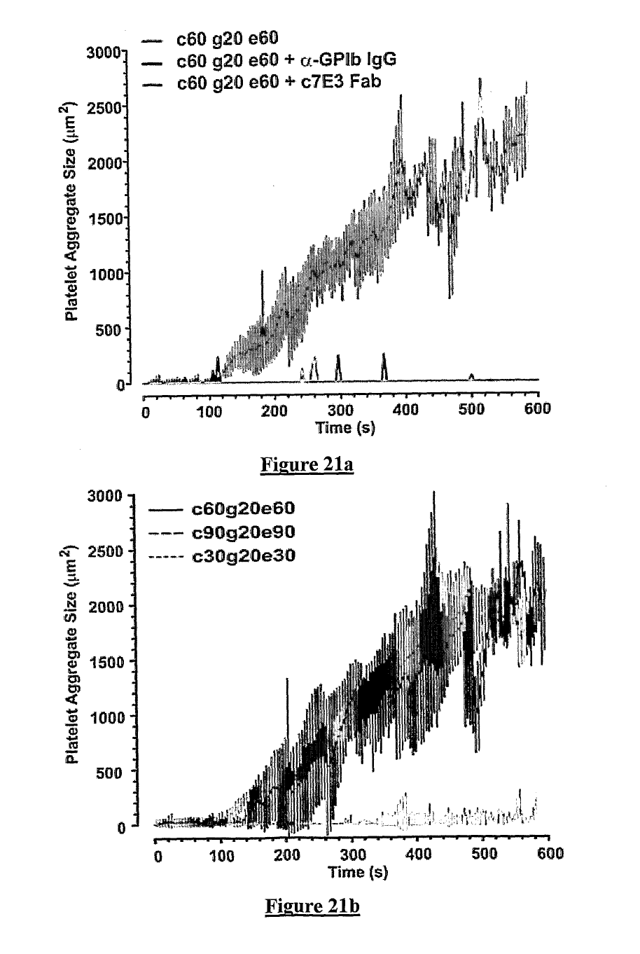

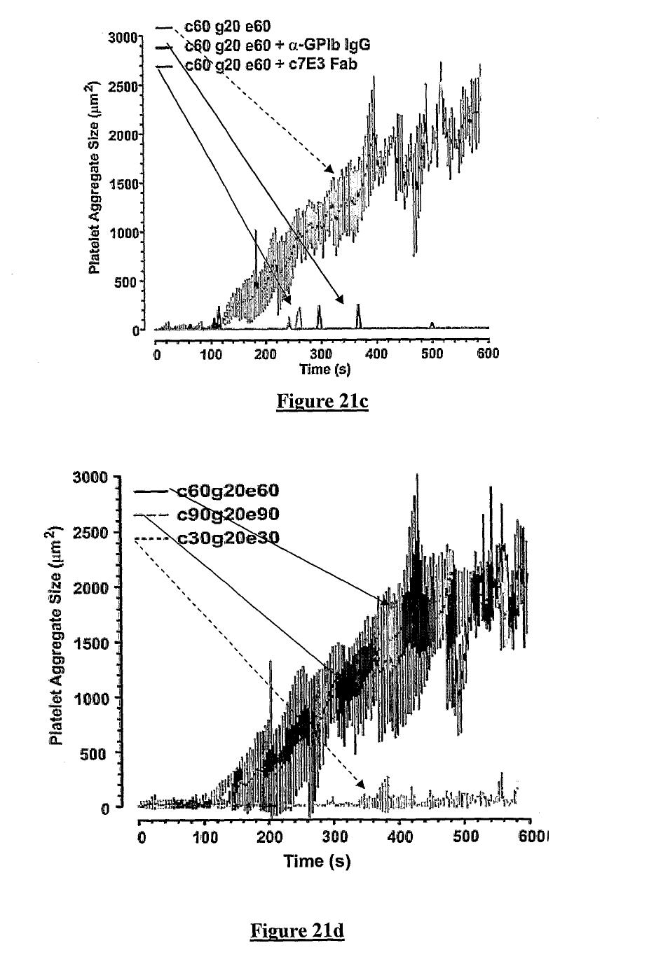

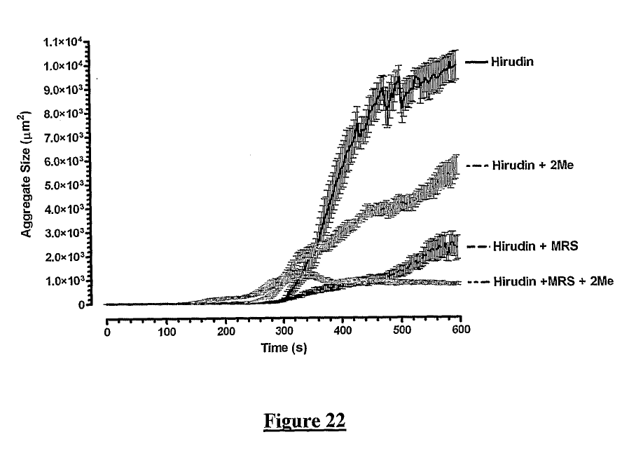

FIG. 20a represents real-time epi-fluorescence imaging of DiOC₆ labeled whole blood perfusion at a constant flow rate of 16 μL/min (input strain rate=1,800 s₋₁) through the c60g20e60 geometric format over a 10 min timeframe, following pre-treatment for 10 minutes with the platelet inhibitors apyrase (0.02 U/ml), N6-methyl-2′-deoxyadenosine-3′,5′-bisphosphate (MRS2179 at 100 μM) and 2-methylthio-AMP (2-MeSAMP at 10 μM) to block ADP; Indomethacin (10 μM) to block TXA₂; and hirudin (800 U/ml) to block thrombin. Perfusion through the c60g20e60 microchannel format resulted in robust platelet aggregation that initiated specifically at the downstream edge of the peak shear (contraction) zone (FIG. 20a). Significantly, platelet aggregation occurred progressively within the downstream strain rate deceleration (expansion) zone resulted in the formation of a relatively large and stable platelet aggregate. Comparison across three independent donor samples showed tight agreement in terms of overall aggregation dynamics and time to occlusion. To investigate the platelet adhesion receptors mediating the aggregation response, whole blood samples were pretreated with the anti-integrin α_{IIb}β₃ ab c7E3 (20 μg/ml) to block the platelet integrin α_{IIb}β₃ or the anti-GPIb IgG Alma12 (50 μg/ml) to block platelet GPIb/V/IX engagement of VWF. As illustrated in FIG. 21a, in the presence of either integrin or GPIb blockade, platelet aggregation within the c60g20e60 geometry was completely inhibited, demonstrating a critical requirement for these primary platelet adhesion receptors in the aggregation process.

### Example 7

### Modulation of Platelet Aggregation as a Function of Microchannel Geometry

As discussed in the preceding, a chief aim of the investigators device design concept was the ability to controllably modulate platelet aggregation by modifying key geometric parameters and therefore the magnitude and extent of the imposed strain rate micro-gradient. FIGS. 21a and 21b show a series of test-case experiments in which both the contraction and expansion angles of the microchannel geometry were symmetrically modified. Comparison of the c60g20e60 geometry format with a c90g20e90 geometry format demonstrated no appreciable difference in the overall magnitude of platelet aggregation, where the input pre-stenosis strain rate was kept constant at 1,800.s⁻¹ (FIG. 21b). However, the c90g20e90 geometry did result in an increased stability of the formed aggregates highlighted by the overall reduction in the variation of aggregate size over time (FIG. 21b). In contrast, a reduction in contraction and expansion angles from 60° to 30° (c30g20e30 format) significantly reduced both the initial rate and magnitude of platelet aggregation (FIGS. 21a & 21b). Interestingly, the site of initial platelet aggregation in the c30g20e30 format was shifted downstream from the stenosis apex, suggesting that an overall strain rate deceleration must be achieved before significant stabilization of platelet aggregation can occur (FIGS. 21a and 21b).

This proof of concept study clearly demonstrated that modification of the strain rate geometry and resultant strain rate distribution in the investigators prototype device can be directly used to modulate platelet aggregation dynamics in a controlled way. Based on the investigators current working hypothesis and the investigators detailed CFD simulations, the inability of the c30g20e30 format to support stable platelet aggregation could be explained by the overall higher strain rates experienced by the developing aggregate (as it is forced to develop within higher velocity regions of the flow) and the overall reduction in the rate of change in strain rate within the expansion zone. In contrast, the increase in aggregate stability in the c90g20e90 format could be explained by more rapid strain rate deceleration and the protection of the formed aggregate from the higher velocity regions of the flow.

In more detail, FIG. 21 a shows representative epifluorescence image sequences of blood perfusion through the c90g20e90 and c30g20e30 microchannel formats. Note that in all cases the blood samples were pretreated with amplification loop blockers (ALB); apyrase (0.02 U/ml), MRS2179 (100 μM) and 2-MeSAMP (10 μM); Indomethacin (10 μM) and hirudin (800 U/ml) for 10 min prior to perfusion (representative of n=3 experiments for each mimetic).

FIG. 21b shows representative aggregation traces showing the response of ALB treated whole blood perfusion through the c60g20e60, c90g20e90 and c30g20e30 microchannel formats (n=3 experiments).

### Example 8

### Comparison of Anti-Platelet Inhibitor Effects in the Microchannel Array

The present example, describes the effect on platelet aggregation of various individual anti-platelet drugs, or combinations of anti-platelet drugs that target specific platelet receptor activation pathways as demonstrated using one iteration of the micro-geometry design. The anti-platelet drugs investigated are ADP receptor/P2Y₁₂ antagonists. ADP is one of the granules released by activated platelets which, in turn activate additional platelets. The granules' contents activate a G_{q}-linked protein receptor cascade, resulting in increased calcium concentration in the platelet's cytosol.

The micro-geometry device used in the present example has a contraction angle (θ_{c}) of 85°, an expansion angle (θₑ) of 85°, a gap width of 30 μm, a gap length of 15 μm and channel entry and exit width of 100 μm (c85 g30 e85 100-100 μm format).

The following anti-platelet drugs were used either alone or in combination:
1. Hirudin: Human whole blood anti-coagulated with Hirudin (800 U/ml) as a control.
2. Hirudin+MRS: Human (hirudin anti-coagulated) whole blood pre-treated for 10 mins with 100 μM of the P2Y₁ adenosine-5′-diphosphate (ADP) antagonist N6-methyl-2′-deoxyadenosine-3′,5′-bisphosphate (MRS2179).
3. Hirudin+2Me: Human (hirudin anti-coagulate) whole blood pre-treated for 10 mins with 10 μM of the P2Y₁₂ (ADP) antagonist 2-methylthio-AMP (2MesAMP).
4. Hirudin+MRS+2Me: Human (hirudin anticoagulated) whole blood pre-treated for 10 mins with the P2Y₁ (ADP) and P2Y₁₂ (ADP) antagonists MRS2179 (100 μM) and 2MesAMP (10 μM).

The data is shown in FIG. 22 and demonstrates that an inhibitor of the P2Y₁₂ (ADP) receptor (the experimental equivalent of clopidogrel (Plavix®)) leads to a 50% reduction in overall aggregation in the device.

The P2Y₁ (ADP) receptor blocker MRS has a much more marked effect on the aggregation profile in the device while in combination, aggregation is severely depressed. The efficacy of the inhibitors appears to be dependent on the type of geometry utilised. For example, when a micro-geometry exhibiting a contraction angle of 90°, an expansion angle of 60° and gap width of 10 μm and entry and exit segments of the microchannel set at 100 and 700 μm respectively (c90 g10 e60 100-700 μm format), the platelet aggregation was resistant to these inhibitors. This data is demonstrated in FIG. 11e where the combined effects of P2Y₁, P2Y₁₂, thrombin and TXA2 inhibitors where the ADP antagonists were used at the same concentration as above, had no effect on the aggregation response.

This data demonstrates that the platelet aggregation response can be specifically customised by changing the angular and contraction dimensions. This allows for a number of device designs that could be utilised to assess different anti-platelet drugs in the clinical setting.

### Example 9

### Comparison of a Normal Healthy Donor Blood Sample vs von Willebrand Disease Blood Sample

The present example demonstrates proof-of concept that the micro-geometry device can be used to differentiate between a blood sample derived from a normal donor versus a blood sample derived from a patient having type III von Willebrand (vWB) disease whose clinically measured vWF blood levels at the time of assay were 7% of normal. von Willebrand disease is the most common hereditary bleeding disorder and is characterised as being inherited autosomal recessive or dominant. In this disease there is a defect in vWF, which mediates the binding of glycoprotein Ib (GPIb) to collagen. This binding helps mediate the activation of platelets and formation of primary hemostasis.

A microchannel geometery comprising a contraction angle (θₐ) of 85°, an expansion angle (θ_{b}) of 85°, a gap width of 30 μm, a gap length of 15 μm and channel width of 100 μm (c85 g30 e85 100-100 μm format) was used.

A health blood sample pre-treated with Hirudin and various anti-platelet drugs was compared with the von Willebrand disease sample pre-treated with Hirudin as various anti-platelet drugs as follows:
Control: Human (hirudin anticoagulated) whole blood pre-treated for 10 mins with the P2Y₁ (ADP) and P2Y₁₂ antagonists MRS2179 (100 μM) and 2MesAMP (10 μM) and the thromboxane A2 inhibitor, Indomethacin (10 μM).
vWD: vonWillebrand disease patient sample (hirudin anti-coagulated) whole blood pre-treated for 10 mins with the P2Y₁ (ADP) and P2Y₁₂ antagonists MRS2179 (100 μM) and 2MesAMP (10 μM) and Indomethacin (10 μM).

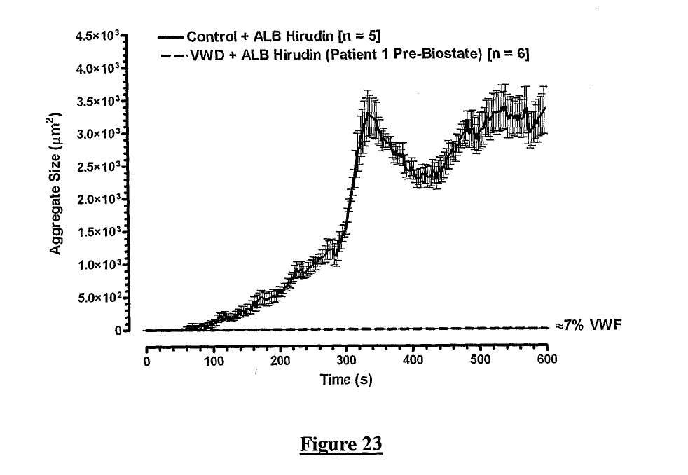

The data is shown in FIG. 23 and demonstrates that at this vWF level, the blood sample from the von Willebrand disease patient is incapable of aggregating in the device containing the above geometry.

### Example 10

### Comparison of Decreasing Contraction Angle on the Platelet Aggregation Response

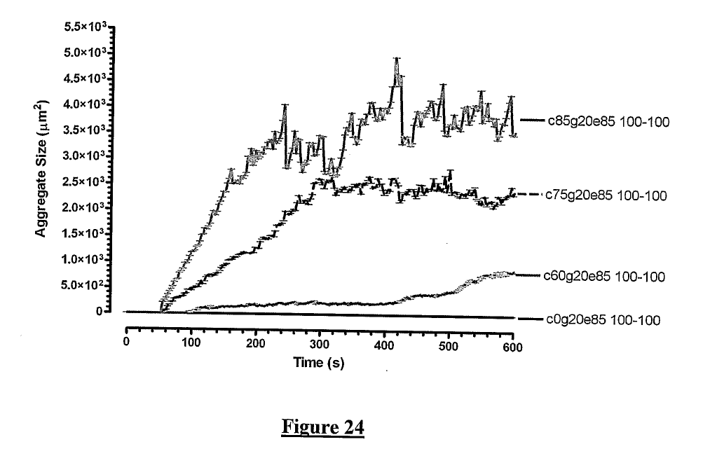

This example explores the role that the contraction (acceleration) angle plays in one iteration of the device. The device was comprised of a gap width of 20 μm, a gap length of 15 μm, an expansion (deceleration angle) of 85° and microchannel entry and exit width of 100 μm (cX g20 e85 100-100 μm format, where cX=contraction angle). Human whole blood was pre-treated for 10 mins with hirudin 800 U/ml and the P2Y₁ (ADP) and P2Y₁₂ antagonists MRS2179 (100 μM) and 2MesAMP (10 μM) respectively and Indomethacin (10 μM). Samples were perfused through the device in which the contraction angle was varied from 0, 60, 75 and 85°. In this iteration, aggregation was effectively eliminated when the contraction angle fell below 60° (see FIG. 24).

### Example 11

### Comparison of Decreasing Expansion Angle on the Platelet Aggregation Response

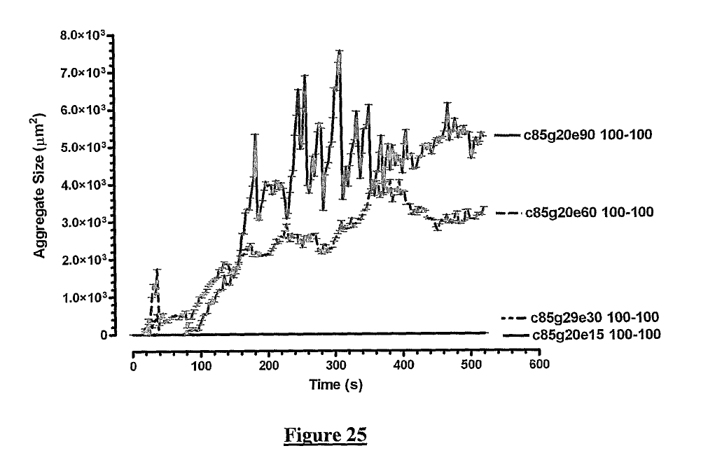

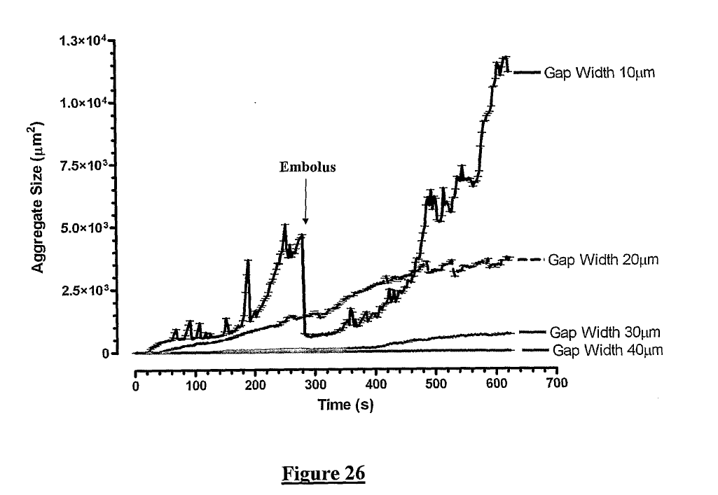

This example explores the role that the expansion (deceleration) angle plays in one iteration of the device. This iteration was comprised of a gap width of 20 μm, a gap length of 15 μm, a contraction (acceleration angle) of 85° and microchannel entry and exit width of 100 μm (c85 g20 eX 100-100 μm format; where eX=expansion angle). Human whole blood was pre-treated for 10 mins with hirudin 800 U/ml and the P2Y₁ (ADP) and P2Y₁₂ antagonists MRS2179 (100 μM) and 2MesAMP (10 μM) respectively and Indomethacin (10 μM). Samples were perfused through the device in which the expansion angle was varied from 15, 60, 75 and 90°. In this iteration aggregation was effectively eliminated when the expansion angle fell below 30° (see FIG. 25).

### Example 12

### Analysis of the Gap Width of the Platelet Aggregation Response

This example demonstrates the role that gap width and therefore the peak shear component plays in the aggregation response in one iteration of the device. This iteration was comprised of a contraction angle of 75°, an expansion angle of 75°, and microchannel entry and exit width of 100 μm (c75 g20 gX e75 100-100 μm format; where gX=variable gap width). Human whole blood was pre-treated for 10 mins with hirudin 800 U/ml and the P2Y₁ (ADP) and P2Y₁₂ antagonists MRS2179 (100 μM) and 2MesAMP (10 μM) respectively and Indomethacin (10 μM). Samples were perfused through the device in which the gap with was varied from 10, 20, 30 and 40 μm. The data demonstrates that the rate and extent of aggregation can be modified by narrowing the gap between the range of 30-10 μm. Platelet aggregation ceases when the gap width drops below 30 μm (see FIG. 26).

### Example 13

### Analysis of the Gap Length on Platelet Aggregation Response

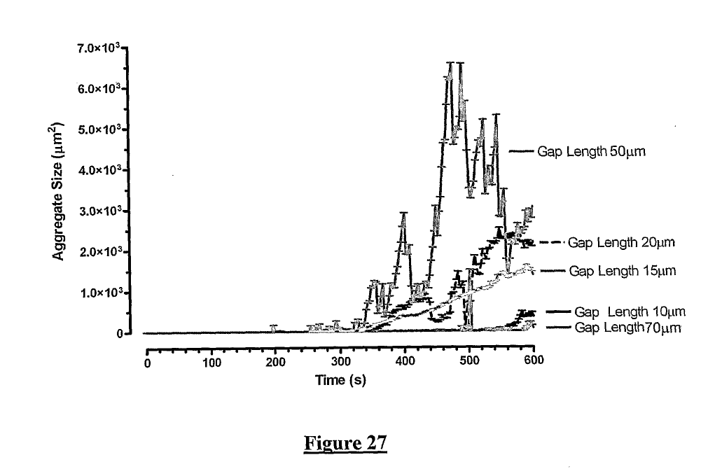

This example demonstrates the role that gap length and therefore the duration of the peak shear component plays in the aggregation response in one iteration of the device. This iteration was comprised of a contraction angle of 75°, an expansion angle of 75°, and microchannel entry and exit width of 100 μm (c75 g20 e75 100-100 μm format; where gap length is varied from 10, 15, 20, 50 and 70 μm). Human whole blood was pre-treated for 10 mins with hirudin 800 U/ml and the P2Y₁ (ADP) and P2Y₁₂ antagonists MRS2179 (100 μM) and 2MesAMP (10 μM) respectively and Indomethacin (10 μM). Samples were perfused through the device in which the gap length was varied between 10 and 70 μm. The data demonstrates that aggregation ceases when the gap length is shorter than 10 μm and also when the gap length exceeds 70 μm. Furthermore the data set demonstrates that the rate and extent of aggregation can be modified by changing the gap length within the 15-50 μm range (see FIG. 27).

1. A microfluidics device to provide real time monitoring of platelet aggregation of a biological sample obtained from a subject, the device comprising:
a channel configured for passage of the biological sample, the channel comprising a protrusion configured to induce an upstream region of shear acceleration coupled to a downstream region of shear deceleration and defining there-between a region of peak rate of shear, the downstream region of shear deceleration defining a zone of platelet aggregation; and
platelet detection means for detecting aggregation of platelets in the zone of aggregation as a result of passage of the biological sample through the channel.

2. The microfluidics device according to claim 1, wherein the protrusion is configured to induce a peak rate of shear within the range 10×10³ s⁻¹ to 150×10³ s⁻¹, when the biological sample is pumped through the device at a rate which defines and constrains initial shear rates to the physiological range (150-10,000 s⁻¹).

3. The microfluidics device according to claim 1, wherein the protrusion comprises an upstream face which is at an angle of between 0° to 90° to a dominant direction of flow through the channel to define the region of shear acceleration, and a downstream face which is at an angle of between 0° to 90° to a dominant direction of flow through the channel to define the region of shear deceleration.

4. The microfluidics device according to claim 3, wherein the upstream face and downstream face are respectively at an angle of between 30° to 90° to a dominant direction of flow through the channel.

5. The microfluidics device according to claim 3, wherein the region of peak shear is defined by a gap width with respect to the protrusion and an opposite channel wall, and the gap width is selected from the range 10 μm to 40 μm.

6. The microfluidics device according to claim 5, wherein a width of the gap, measured parallel to a dominant direction of flow through the channel, is between 0.5 and 20 μm.

7. A The microfluidics device according to claim 3, wherein the upstream and downstream faces are substantially planar, concave or convex.

8. A microfluidics device for assessing platelet aggregation of a biological sample obtained from a subject, the device comprising:
a channel configured for passage of the biological sample, the channel having a protrusion for perturbing flow of the sample, at least one cross-sectional dimension of the protrusion being less than substantially 100 micrometres, and the protrusion being configured to define a zone of platelet aggregation within the channel; and
platelet detection means for detecting aggregation of platelets at the zone of aggregation as a result of passage of the biological sample through the channel.

9. The microfluidics device according to claim 8, wherein the channel configuration and flow rate are adapted to maintain Reynolds numbers within the channel less than or equal to about 26, in order to maintain fully stable blood flow without flow separation or vortex formation.

10. The microfluidics device according to claim 8, wherein the protrusion comprises a spherical protrusion located within the channel around which the sample must flow.

11. The microfluidics device according to claim 10, wherein the spherical protrusion is centrally located across a width of the channel such that substantially equal amounts of the sample flow on each side of the spherical protrusion.

12. The microfluidics device according to claim 8, wherein a plurality of channels are provided, each channel having a protrusion of substantially the same dimensions, and wherein the detection means is operable to detect a sum of all platelet aggregation in all the channels.

13. The microfluidics device according to claim 1, wherein a plurality of channels are provided, each channel having a protrusion of substantially different dimensions, and wherein the detection means is operable to detect in parallel, differential platelet aggregation in the array of channels.

14. The microfluidics device according to claim 8, wherein the channel surface is provided with a serum protein, an adhesive substrate or a polymer in order to improve platelet aggregation.

15. The microfluidics device according to claim 8, wherein the platelet detection means comprises an optical detection means.

16. The microfluidics device according to claim 15, wherein the optical detection means comprises a total internal reflection sensor which is situated adjacent the channel protrusion to monitor real-time platelet aggregation in the zone of platelet aggregation.

17. The microfluidics device according to claim 15, wherein the optical detection means comprises a light emitter and an aligned light detector, wherein the light emitter is configured to emit light for internal reflection within a material from which the channel is formed, such that the light detector detects changes in internal light reflection brought about by aggregation of platelets in the zone of platelet aggregation.

18. The microfluidics device according to claim 15, wherein the optical detection means comprises a light emitter and an aligned light detector, and the light emitter is configured to emit light for transmission through the zone of platelet aggregation such that the light detector detects a reduction in transmitted light intensity brought about by aggregation of platelets.

19. The microfluidics device according to claim 15 wherein the optical detection means comprises a light emitter and an aligned light detector, and the light emitter is configured to emit light through a zone of platelet aggregation of each of a plurality of channels as defined by respective protrusions, such that the light detector may detect a reduction in transmitted light intensity brought about by total platelet aggregation in all channels.

20. The microfluidics device according to claim 15, wherein the device comprises a fabricated block within which are formed, embedded or moulded, one or more fluid-tight channels.

21. The microfluidics device according to claim 20, wherein the block material from which the device is fabricated is one of Polydimethylsiloxane (PDMS), borosilicate glass, SF11 glass, SF12 glass, polystyrene and polycarbonate.

22. A diagnostic method comprising:
passing a bilogical sample through a icrofluidics device comprising:
a channel configured for passage of the biological sample, the channel comprising a protrusion configured to induce an upstream region of shear acceleration coupled to a downstream region of shear deceleration and defining there-between a region of peak rate of shear, the downstream region of shear deceleration defining a zone of platelet aggregation; and
platelet detection means for detecting aggregation of platelets in the zone of aggregation as a result of passage of the biological sample through the channel; and
providing an indication of the detected aggregation of platelets.

23. The method of claim 22, comprising:
i) passing the biological sample through the microfluidics device under defined flow conditions and for a time sufficient to enable cells from the biological sample to aggregate;
ii) detecting any aggregation of said cells; and
iii) comparing the time to and size of the aggregation of cells of the biological sample with a predetermined standard, wherein any variation is indicative of the presence of or risk of developing a condition or disorder involving abnormal function or activity of platelets or their progenitors.

24. The method of claim 22, comprising:
i) passing said biological sample in the presence of said reagent(s) through the microfluidics device, under defined flow conditions and for a time sufficient to determine whether platelet aggregation has occurred within said device; and
ii) comparing the result obtained in step (i) with the result when step (i) is performed in the absence of said reagent(s).

25. The method of claim 22, comprising:
(i) passing a first biological sample from the subject through the microfluidic device under defined flow conditions and for a time sufficient to determine whether platelet aggregation has occurred within said device, said first biological sample being obtained prior to administration of the reagent to the subject, and
(ii) passing a second biological sample from the same subject through the microfluidic device, under defined flow conditions and for a time sufficient to determine whether platelet aggregation has occurred within said device, said second biological sample being obtained after administration of the reagent to the subject; and
(iii) comparing the result obtained in step (i) with the result obtained in step (ii).

26. The method of claim 22, comprising:
(i) passing a first biological sample from the subject through the microfluidic device under defined flow conditions and for a time sufficient to determine whether platelet aggregation has occurred within said device, said first biological sample being obtained after a first dose of the reagent to the subject, and
(ii) passing a second biological sample from the same subject through the microfluidic device, under defined flow conditions and for a time sufficient to determine whether platelet aggregation has occurred within said device, said second biological sample being obtained after a second dose of the reagent to the subject; and
(iii) comparing the result obtained in step (i) with the result obtained in step (ii).

27. The method of claim 22, comprising using the indication of the detected aggregation of the platelets to monitor platelet function and/or viability in a biological sample.

28. The method of claim 22, comprising
(i) contacting at least one biological sample obtained from a subject with at least a first member of a plurality of candidate anti-platelet compounds;
(ii) passing the at least one sample through the microfluidics device according to claim 1, under defined flow conditions and for a time sufficient to determine whether platelet aggregation has occurred within said device;
(iii) detecting an effect of the first member of the plurality of candidate anti-platelet compounds on the platelet aggregation of the at least one biological sample; and
(iv) comparing the effect observed in (iii) with a control sample that has not come into contact with the candidate compound.

29. The method of claim 23, comprising providing an anti-platelet reagent selected using the effect or the comparison.

30. A kit for use in monitoring platelet function, comprising packaging material comprising:
(i) a microfluidics device; and
(ii) instructions for indicating that the microfluidics device is to be used in a system for monitoring platelet function; and
wherein the microfluidics device comprises:
a channel configured for passage of the biological sample, the channel comprising a protrusion configured to induce an upstream region of shear acceleration coupled to a downstream region of shear deceleration and defining there-between a region of peak rate of shear, the downstream region of shear deceleration defining a zone of platelet aggregation; and
platelet detection means for detecting aggregation of platelets in the zone of aggregation as a result of passage of the biological sample through the channel.

31. The method of claim 22, comprising:
passing the biological sample through a featured channel at a rate which causes the channel featuring to perturb flow of the sample so as to induce an upstream region of shear acceleration coupled to a downstream region of shear deceleration and defining there-between a region of peak rate of shear, the downstream region of shear deceleration defining a zone of platelet aggregation; and
detecting, in real time, aggregation of platelets in the zone of aggregation as a result of passage of the biological sample through the channel.
